(19) United States
(12) Patent Application Publication
(10) Pub. No.: US 2026/0248804 A1
(43) Pub. Date: Aug. 27, 2026

(54) Pharmacotherapy for Opioid Tapering

(71) Applicant: Northwestern University, Evanston, IL (US)

(72) Inventors: Apkar Vania Apkarian, Chicago, IL (US); Marwan N. Baliki, Chicago, IL (US)

(73) Assignee: Northwestern University, Evanston, IL (US)

(21) Appl. No.: 19/551,323

(22) Filed: Feb. 26, 2026

Related U.S. Application Data
(60) Provisional application No. 63/764,388, filed on Feb. 27, 2025.

(51) Int. Cl. A61K 31/541; A61B 5/00; A61K 31/422; A61K 31/485; A61K 31/495; A61K 31/496; A61K 31/506; G16H 10/60; G16H 20/10

(52) U.S. Cl. CPC A61K 31/541; A61B 5/4848; A61K 31/422; A61K 31/485; A61K 31/495; A61K 31/496; A61K 31/506; G16H 10/60; G16H 20/10

(57) ABSTRACT

Methods and compounds to therapeutically treat a molecular target to counteract circuit adaptations of long-term opioid use that are associated with a functional disability and negative affect in both CBP−O (chronic back pain managed without opioids) and CBP+O (chronic back pain managed with opioids).

### RELATED APPLICATION DATA

This application claims the benefit of U.S. Provisional Application No. 63/764,388, filed Feb. 27, 2025, which is hereby incorporated by reference in its entirety.

### STATEMENT OF GOVERNMENT INTERESTS

This invention was made with government support under grant number DA044121 awarded by the National Institutes of Health and grant number W81XWH-17-1-0426 awarded by the Department of Defense. The government has certain rights in the invention.

### FIELD

The present disclosure relates generally to methods of tapering long-term opiate use for pain management. In particular, methods of exploiting molecular targets via pharmacotherapies to aid in opioid tapering are disclosed herein.

### BACKGROUND

Millions of chronic (non-cancer) pain patients manage their pain with long-duration opioid use [1-3], but the neurobiological implications of stable long-term opioid consumption remain unknown. In the present disclosure, the clinical profiles and brain structure and function of 70 chronic back pain patients prescribed opioids (CBP+O [chronic back pain managed with opioids], average opioid exposure of 7.8 years) were contrasted with 70 matched patients managing their pain without opioids (CBP−O [chronic back pain managed without opioids]) and 39 matched healthy subjects. Despite CBP+O exhibiting only modestly worse clinical profiles than CBP−O with small differences in brain morphology, CBP+O had starkly different brain activity (ALFF). These brain activity differences strongly reflected the cortical spatial distributions of serotonin (5-HT_{1A} and 5-HT_{1B}) and muopioid (MOR) receptor densities: CBP+O had greater MOR- and lower serotonin-related activity. Serotonin and MOR-related activities were associated with patients' functional disability, negative affect, opioid dose, abstinence status, and, in an independent sample, tapering success, indicating that receptor-related activity is a biomarker for multiple clinically relevant characteristics of CBP+O patients. Through pharmacological perturbations in two separate studies (opioid abstinence and 5-HT_{1A} agonist administration), it was found that cortical serotonergic and opioidergic circuits comprise opponent processes. The methods disclosed herein that show that long-term opioid use implicates multiple receptors and their interaction points to a molecular target and a molecule that may be exploited to aid opioid tapering.

### SUMMARY

This Summary introduces a selection of concepts relating to this technology in a simplified form as a prelude to the Detailed Description that follows. This Summary is not intended to identify key or essential features.

In some aspects, a method for facilitating opioid tapering in a patient undergoing long-term opioid therapy may include the steps of administering a therapeutically effective amount of a pharmaceutical composition targeting serotonergic and opioidergic receptor activity, modulating serotonin (5-HT1A, 5-HT1B) and mu-opioid receptor (MOR) activity to counteract opioid-induced neuroadaptive changes, monitoring a patient's neurobiological response to the pharmaceutical composition, and adjusting the administering of the pharmaceutical composition based on a response of the patient to facilitate opioid dose reduction while minimizing withdrawal symptoms and pain exacerbation. In some examples, the pharmaceutical composition may include a selective 5-HT1A agonist, a 5-HT1B modulator, or combinations thereof. In other examples, the method may include a mu-opioid receptor partial agonist or antagonist to modulate opioid receptor desensitization and withdrawal effects. In one example, the pharmaceutical composition may be administered in a controlled tapering protocol over a predetermined period. In yet another example, the method may further include assessing cortical receptor-related activity before and during the opioid tapering process using functional neuroimaging.

In other aspects, a pharmaceutical composition for aiding opioid tapering disclosed herein may include a therapeutically effective amount of a 5-HT1A agonist, a therapeutically effective amount of a 5-HT1B modulator, a mu-opioid receptor partial agonist or antagonist, and a pharmaceutically acceptable carrier. In some examples, the 5-HT1A agonist may be buspirone, flesinoxan, or an equivalent compound. In another example, the 5-HT1B modulator may be zolmitriptan, elzasonan, or an equivalent compound. In still other examples, the mu-opioid receptor partial agonist or antagonist may be buprenorphine, nalmefene, or an equivalent compound.

In one aspect, a method of identifying patients responsive to opioid tapering treatment disclosed herein may include the steps of measuring baseline cortical receptor activity using neuroimaging techniques, administering a test dose of a 5-HT1A agonist, monitoring changes in cortical receptor-related activity, correlating changes with clinical opioid withdrawal severity, and adjusting opioid tapering protocols based on a patient neurobiological response profile. In one example, the neuroimaging techniques may include resting-state functional MRI, positron emission tomography, or electroencephalography.

In another aspect disclosed herein, a method of mitigating opioid withdrawal symptoms may include the steps of co-administering a serotonergic agent and a mu-opioid receptor modulator during opioid tapering to reduce withdrawal severity and enhance treatment adherence.

In one aspect disclosed herein, a method of preventing opioid-induced hyperalgesia during tapering may include administering a serotonergic agent to counteract opioid withdrawal-induced hyperexcitability in cortical pain-processing circuits.

In still other aspects disclosed herein, a system for guiding opioid tapering may include a database containing neuroimaging profiles of patients undergoing opioid tapering, a processing unit configured to analyze receptor-related activity and predict optimal tapering protocols, and a user interface for clinicians to input patient-specific data and receive personalized tapering recommendations. In some examples, the processing unit may employ machine learning algorithms to refine opioid tapering strategies based on patient response patterns over time.

In yet other aspects disclosed herein, a pharmaceutical composition for facilitating opioid tapering in patients undergoing long-term opioid therapy may include a therapeutically effective amount of a serotonin receptor modulator, a therapeutically effective amount of an opioid receptor modulator, and a pharmaceutically acceptable carrier. In some examples, the serotonin receptor modulator may be a 5-HT1A agonist selected from the group consisting of vortioxetine, buspirone, flesinoxan, and tandospirone. In one example, the opioid receptor modulator may be a partial agonist or antagonist selected from the group consisting of buprenorphine, nalmefene, and naltrexone. In yet another example, the pharmaceutical composition may be formulated for controlled-release administration to sustain therapeutic effects over an extended period.

In still other examples, an administration regimen may be adjusted based on patient-specific factors including opioid dependence severity, withdrawal symptoms, and neurobiological response to treatment.

These and additional features will be appreciated with the benefit of the disclosures discussed in further detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which like reference numerals refer to the same or similar elements in all of the various views in which that reference number appears.

FIGS. 1A-1F graphically depict long-term opioid use is associated with worse functional disabilities and worse pain quality, according to one or more aspects described herein. FIG. 1A plot shows standardized clinical parameter differences (±95% CI) between CBP+O and CBP−O (higher scores corresponding to worse outcomes are shown in red, while lower scores corresponding to worse outcomes are blue). CBP+O showed significantly worse outcomes across most measures. Right three columns: These clinical parameters loaded on three principal components (PC1-3) that explained 69.9% of the variance. Bars represent factor loadings of each measure. Identified PC-s were uniquely associated with functional disability (PC1), pain quality (PC2), and negative affect (PC3). FIG. 1B shows compared to CBP−O, CBP+O exhibited worse functional disability (PC1) and worse pain quality (PC2). FIG. 1C shows distribution of prescription opioid consumption (MME), blood opioid levels (ROE in mg/L), and duration of opioid use (DOU in years) in CBP+O. CBP+O showed a varied and wide range of opioid use, with 31 patients within the no-risk range (MME<20), 21 patients' minimal risk (MME 20-50), 7 patients' moderate risk (MME 50-90), and 5 patients' high-risk range (MME>90). All three outcomes (MME, ROE, DOU) were right-skewed. Therefore, they were log-transformed to conform to normal distributions. FIG. 1D is a heat map that shows the relationship of opioid measures (log) with each other, and with consumption of non-opioid medications (MQS). FIG. 1E shows MME and ROE have a significant relationship (scatter plot). FIG. iF plot shows standardized regression estimates (±95% CI) for the relationship between opioid measures (log) with signs of misuse (COMM) and withdrawal (SOWS) scales, pain intensity, and the three principal components (PC1-3) across all 70 CBP+O. Only opioid blood levels (ROE) showed a significant relationship with PC1-functional disability; higher blood opioids were associated with worse functional disability. PainDETECT=presence of neuropathic pain; MPQ=McGill Pain Questionnaire, subscales are indicated; PCS=Pain Catastrophizing Scale; ODI=Oswestry low back Disability Index; PROMIS=Patient-Reported Outcomes Measurement Information System, subscales are labeled, pain int.=pain interference; SF12=Short Form quality of life scale; BDI=Becks Depression Inventory; PANAS=Positive and Negative Affect Schedule.

FIGS. 1G-1H graphically depict long-term opioid use is associated with worse functional disabilities and worse pain quality, according to one or more aspects described herein. FIG. 1G scatter plots show the relationship of principal components scores determined from CBP−O (PC1′, PC2′, PC3′) with component scores computed from all patients (PC1, PC2, PC3). FIG. 1H scatter plots show the relationship of principal component scores determined from CBP+O (PC1″, PC2″, PC3″) with component scores computed from all patients (PC1, PC2, PC3). Overall, principal components computed from CBP−O and CBP+O showed similar scores to those computed from all patients (all R-values >0.92; p<0.01).

FIG. 1I depicts opioid blood level is positively associated with worse functional disability (PC1). Opioid blood levels (ROE) showed a significant positive correlation and PCl (functional disability) scores corrected for age, sex, NRS, pain duration, BMI, and MQS (p<0.01). BMI=body mass index; MQS=medication quantification scale. NRS=numerical pain rating scale. ROE=relative opioid equivalent in milligrams/liters, according to one or more aspects described herein.

FIGS. 1J-1K depict relationship of pain intensity with pain quality and with consumption of non-opioid medications, in CBP patients, according to one or more aspects described herein. FIG. 1J—PC2 (Pain quality) showed a significant positive relationship with pain intensity for CBP+O and CBP−O patients. FIG. 1J—Compared to CBP−O, CBP+O patients showed increased consumption of non-opioid drugs as assessed using MQS (p<0.001). MQS was related to pain intensity in CBP+O group (p<0.01), but not in the CBP−O group (p=0.97). These results are informative since even when pain intensity and pain duration are matched between CBP+O and CBP−O, and even though the relationship between pain intensity and pain affect are very similar between the two groups, CBP+O consume more non-opioids in proportion to their pain. The latter provides indirect evidence that long-term opioid use exacerbates the affective component of back pain. Thus, demonstrating an opioid-induced hyper-affective state when pain intensity is matched between the groups, indirectly demonstrating the presence of opioid-induced hyperalgesia. MQS=medication quantification scale. NRS=numerical pain rating scale.

FIGS. 2A-2E depict spontaneous activity with long-term opioid use maps to three cortical receptor density distributions, according to one or more aspects described herein.

FIGS. 2F-2G depict long-term opioid use was associated with lower global and local gray matter, according to one or more aspects described herein. FIG. 2F shows peripheral gray matter volume (PGMV) was significantly less in CBP+O compared to CBP−O. FIG. 2G CBP+O showed less gray matter density in SI/Mi and mACC (TFCC t-score >2.3, p<0.01 corrected for multiple comparisons). PGMV=peripheral gray matter volume; S1/ M1=primary sensorimotor cortex; mACC=middle anterior cingulate cortex.

FIG. 2H shows mid-anterior Cingulate Gray matter density was negatively associated with pain log pain duration and pain intensity, in both CBP+O and CBP−O groups. Gray matter density in the mACC was significantly associated with pain duration (left scatter plot) and pain intensity (right scatter plot) in all CBP patients (statistical significance is presented in Table 9). mACC=middle anterior cingulate cortex.

FIGS. 3A-3F depict serotonin (5HT₁A, 5HT₁B), and opioid (MOR) receptor-related whole-cortex activity that are modulated with long-term opioid use and reflect distinct clinical dimensions, according to one or more aspects described herein.

FIGS. 4A-4E depict serotonin (5HT₁A, 5HT₁B), and opioid (MOR) related cortical activity show specific adaptations with opioid exposure manipulations, according to one or more aspects described herein.

FIGS. 5A-5B depict effect of a single dose of 5HT₁A agonist on receptor-related whole-cortex activity (a), and a minimal model of long-term opioid exposure (b), according to one or more aspects described herein.

FIGS. 6A-6C depicts modulation of serotonin and opioid dependent brain activity following long-term fentanyl exposure in mouse model for chronic pain, according to one or more aspects described herein. FIG. 6A—Boxplots display the withdrawal thresholds of the injured paw in SNI and Sham mice at baseline and following long-term exposure to fentanyl. Paw withdraw thresholds were significantly lower in SNI animals compared to sham (P<10-3) and did not change following fentanyl exposure compared to baseline (P=0.84). FIG. 6B—Brain images show regions that showed increased (red-yellow) and decreased (blue-green) spontaneous activity (change in ALFF) following fentanyl exposure in SNI (top row) and Sham (bottom row). FIG. 6D—Scatter plot show relationship between ALFF changes (fentanyl >baseline) with 5-HT_{1A} (left), 5-HT1B (middle) and MOR (right) receptor density for SNI (top row) and sham (bottom row) across 92 brain ROIs. ALFF changes in SNI were negatively correlated with 5-HT_{1B} (P<10-5) and positively associated with MOR (P<10-5). ALFF changes in sham were positively associated with MOR (P<10-5) and not associated with 5-HT_{1A} and 5-HT_{1B}.

Further, it is to be understood that the drawings may represent the scale of different components of various examples; however, the disclosed examples are not limited to that particular scale. Further, the drawings should not be interpreted as requiring a certain scale unless otherwise stated.

### DETAILED DESCRIPTION

Chronic pain afflicts over 20% of the world's population4 and has massive economic and societal ramifications. Debilitating pain can hijack patients' lives, preventing them from freely being able to work, exercise, and socialize. Chronic low back pain (CBP), for example, is the leading cause of disability in the world [5]. Until the mid-2010 s, prescription opioids were commonly used as a first-line treatment for chronic pain, making chronic pain a natural entry point for opioid use and, thus, potential downstream misuse, opioid use disorder (OUD), and even overdose [6-8]. Despite marked decreases in opioid prescription rates within the healthcare system over the past ten years, opioids are still used by 20-50% of patients suffering from chronic (non-cancer) pain (around 15 million patients in the US) [1-3]. The neuropsychological implications of long-term opioid use in patients with chronic pain are largely unknown.

Chronic non-cancer pain (the population of interest throughout this study) and opioids have been extensively studied on their own. Each imparts a massive societal cost [9,10], and their combination may impart an even greater cost. Our group has demonstrated that the development of chronic pain involves mesocorticolimbic (MCL) plasticity [11-16]. Interestingly, these same circuits are strongly implicated in OUD [17,18]. From a high level, MCL helps code emotional valence and modulate behavioral responses, such as appetition and aversion [19-21]. Pain is an aversive experience, and patients actively avoid triggers. In contrast, opioids, while commonly said to be rewarding, may induce aversion and negative affect during periods of withdrawal—so-called hyperkatifeia [22]. As a result, the mechanisms of chronic pain may interact with long-term opioid use, exacerbating patients' pain experience when off opioids to create a vicious cycle. However, there is little data on the impact of long-term opioid consumption in chronic pain patients [23-28].

As disclosed herein, the clinical characteristics, brain anatomy, and brain function of CBP prescribed long-term stable doses of opioids (CBP+O, n=70; mean=7.8 years of opioid consumption) were compared to CBP patients managing their pain without opioids (CBP−O, n=70). The two groups were one-to-one matched (from a150 CBP−O dataset) based on their age, sex, pain intensity (mean=5.4 on a 0-10 numeric rating scale), and pain duration (>0.5 years, mean=17 years) (Table 1).

It was reasoned that long-term opioid use should also lead to a reorganization of the relationship between neurotransmitter receptor-related activity, given that opioid exposure leads to receptor desensitization and tolerance [29], which in turn must interact and modify the influence of other receptors on neuronal activity. Moreover, it was recently showed that perceptual states, especially pain perception, engage neuronal activity throughout the brain [30], prompting development of an approach to examine whole-brain multi-receptor-related adaptations. Therefore, the neurotransmitter atlas recently developed by Hansen et al. [31] was used and related methods were modified to relate whole-cortex activity for long-term opioid exposure to the chemoarchitecture of the human brain, based on the brain normative map for 19 receptors and transporters across 9 different neurotransmitter systems. Receptor-related activity was then compared (i) between subgroups of CBP+O (high and low opioid consumption), (ii) within CBP+O subjects before and after brief opioid abstinence, and (iii) between CBP+O subjects who tapered or did not taper opioid use. With back translational, mouse model (sham or chronic pain) were validated before and after the mice self-administered vaporized fentanyl for multiple weeks. The results disclosed herein show a novel multi-receptor model for long-term opioid use and identify a specific target receptor and a molecule that may aid opioid tapering.

TABLE 1

| | | |
|---|---|---|
| CBP − O (n = 70) | CBP + O (n = 70) | |
| mean ± s.d. | mean ± s.d. | p-value |

Demographic and general health characteristics

| Age (years) | 59.87 ± 11.97 | 60.10 ± 10.50 | | 0.87 |
|---|---|---|---|---|
| Sex/female (%) | 46 (65.71%) | 46 | (65.71%) | 1 |
| Alcohol (%) | 46 (65.71%) | 33 | (47.14%) | 0.06 |
| Smoking (%) | 13 (18.57%) | 15 | (21.42%) | 0.90 |
| Race | | | | |
| White | 41 (58.57%) | 45 | (64.29%) | 0.47 |
| Black | 15 (21.43%) | 23 | (32.86%) | 0.23 |
| Other/Undisclosed | 14 (20.00%) | 2 | (2.86%) | 0.01 |

| | | | |
|---|---|---|---|
| BMI | 30.12 ± 5.21 | 31.16 ± 7.36 | 0.35 |
| MQS | 10.33 ± 7.56 | 20.53 ± 12.35 | <10⁻⁵ |

Pain characteristics

| | | | |
|---|---|---|---|
| Pain intensity (NRS) | 5.34 ± 2.13 | 5.40 ± 1.94 | 0.87 |
| Pain duration (years) | 16.64 ± 19.16 | 17.21 ± 12.36 | 0.68 |

Opioid consumption and behavior

| | | |
|---|---|---|
| MME | — | 41.41 ± 70.06 |
| ROE (mg/L) | — | 0.0042 ± 0.009 |
| Duration of opioid | — | 7.80 ± 6.09 |
| use (years) | | |
| SOWS | | 8.83 ± 8.27 |
| COMM | — | 7.17 ± 6.10 |

As shown above in Table 1, CBP+O and CBP−O patients were matched one-to-one between 70 CBP+O and a select 70 of 150 CBP−O, for age, sex, alcohol use, cigarette smoking, and BMI (two-tailed unpaired t-tests or chi-square tests were performed). They were also matched for pain intensity and duration. Opioid consumption was quantified using the daily prescription converted into MME, the blood levels of opioids converted to an ROE (mg/L), and the duration of opioid use in years. On average, CBP+O exhibited low opioid use (MME<50) and high non-opioid medication use compared to CBP−O. On average opioid blood concentrations were 10 times lower than those usually observed in heroine misuse (around 0.05 mg/L) and more than 25 times less than those detected in heroin overdose death (>0.1 mg/L). The CBP+O patient with the highest opioid blood level displayed a ROE value of 0.048 mg/L that corresponded to the second highest MME value of 210. The overall low opioid use in the patients was consistent with the observed low withdrawal symptoms (SOWS) and misuse (COMM). There was no relationship between pain duration and duration of opioid use in CBP+O. Data presented as mean±s.d. BMI=body mass index; MQS=medication quantification scale. NRS=numerical rating scale; MME=daily morphine milligram equivalent; ROE=relative opioid equivalent. SOWS=subjective opioid withdrawal scale; COMM=current opioid misuse measurement.

Chronic pain and opioid use are independently associated with multiple psychological comorbidities, including worse negative affect, sleep disturbance, and diminished social interactions [8,18,32]. Currently, there is little data to answer if these outcomes are worse in patients managing their chronic pain with long-term opioid consumption [23,33-35]. There is clinical equipoise: On one hand, prescription opioids may improve patients' psychological characteristics via pain relief. On the other hand, if prescription opioids enhance the likelihood of OUD, they may exacerbate rather than relieve the psychological challenges associated with chronic pain, for example, prescription opioids are associated with worse depression [36].

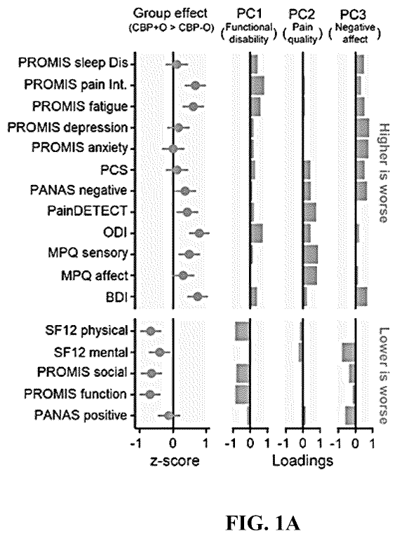

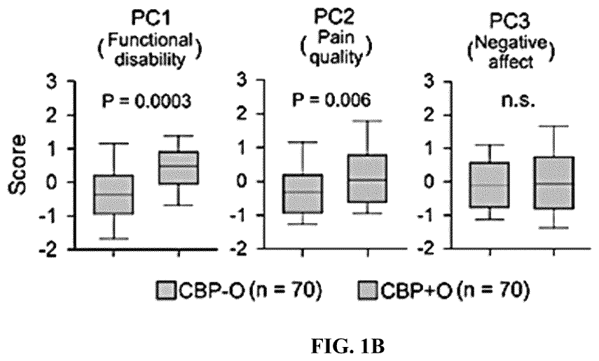

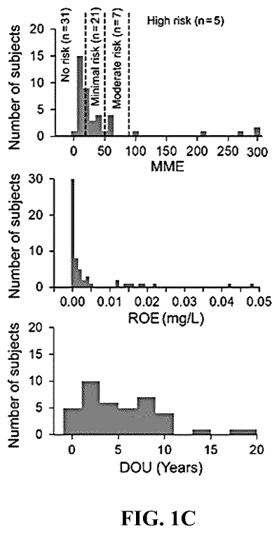

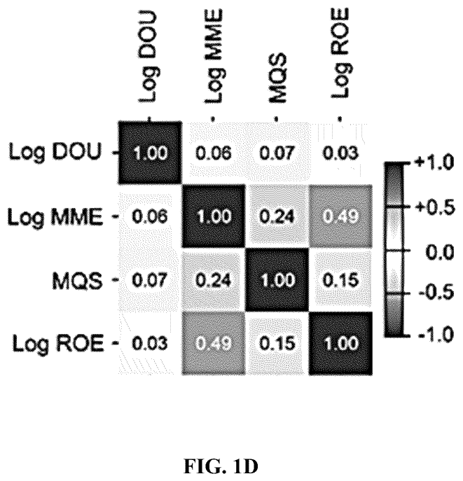

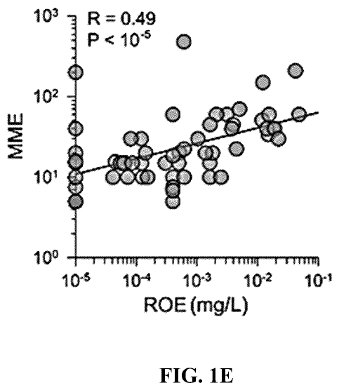

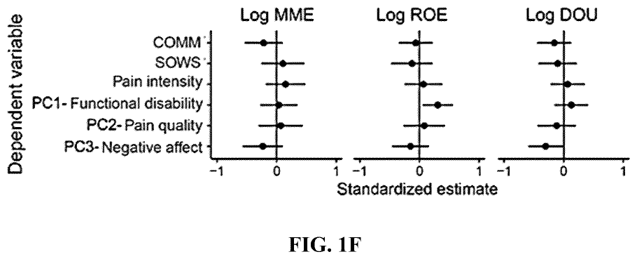

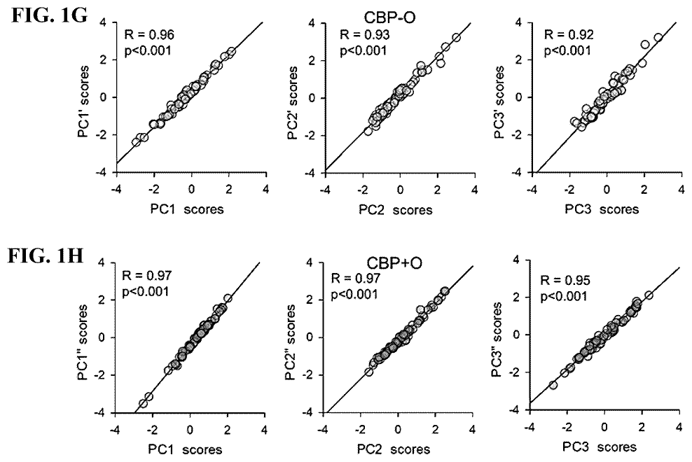

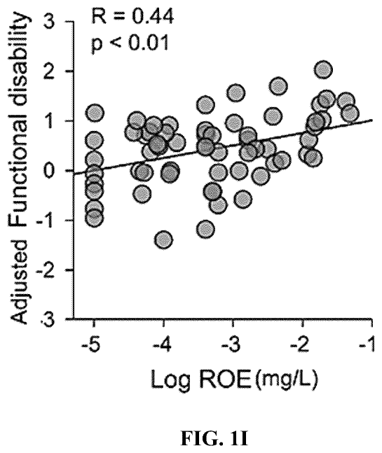

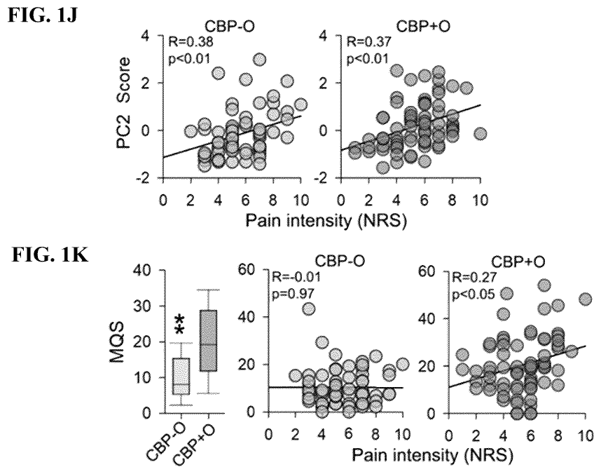

Using validated self-report questionnaires, physical function, pain, and mental health were compared between CPB+O and CBP−O. Compared to CBP−O, CBP+O exhibited worse outcomes for 7 of the 17 measures; only anxiety (PROMIS) was similar, and all other outcomes tended to show worse values in CBP+O relative to CBP−O, albeit with appreciable uncertainty (see FIG. 1A, Table 2). A principal components analysis (PCA) was conducted to reduce the dimensionality of these outcomes. Three principal components (PC1-3; variance >10%) explained 69% of the total variance: PCl (called functional disability) included decreased physical function, less social activity, increased general disability, greater pain interference, and greater fatigue; PC2 (pain quality) was mainly composed of pain descriptors, including sensory and affective pain scales, neuropathic pain symptoms, and pain catastrophizing; PC3 (negative affect) included increased depression, anxiety, negative affect, decreased mental well-being, and less sleep (FIG. 1A, Table 3). These PCs were stable no matter how they were derived (FIGS. 1G-1H, Table 3) and closely resemble the results previously reported [23].

TABLE 2

| | CBP − O | CBP + O | | |
|---|---|---|---|---|
| | (n = 70) | (n = 70) | t-score | |
| | mean ± s.d. | mean ± s.d. | (p-value) | |
| PainDETECT | 11.78 ± 7.56 | 15.06 ± 7.53 | −2.57 | (0.01) |
| MPQ sensory | 11.48 ± 6.84 | 14.87 ± 6.75 | −2.94 | (<10⁻²) |
| MPQ affect | 2.89 ± 2.67 | 3.81 ± 3.33 | −1.82 | (0.07) |
| PCS | 13.27 ± 10.09 | 14.53 ± 11.50 | −0.68 | (0.49) |
| ODI | 30.22 ± 13.02 | 41.42 ± 13.17 | −5.06 | (<10⁻⁵) * |
| PROMIS function | 41.12 ± 6.51 | 36.59 ± 5.72 | 4.35 | (<10⁻⁵) * |
| PROMIS anxiety | 50.71 ± 8.77 | 50.72 ± 8.89 | −0.01 | (0.99) |
| PROMIS depression | 47.64 ± 7.89 | 48.93 ± 8.52 | −0.94 | (0.35) |
| PROMIS fatigue | 51.44 ± 8.22 | 56.69 ± 8.30 | −3.76 | (<10⁻³) * |
| PROMIS sleep dis | 53.21 ± 8.21 | 54.26 ± 10.35 | −0.67 | (0.51) |
| PROMIS social act | 49.15 ± 8.70 | 43.82 ± 7.80 | 4.04 | (<10⁻⁴) * |
| PROMIS pain Int. | 58.98 ± 6.26 | 63.19 ± 5.61 | −4.18 | (<10⁻⁴) * |
| SF12 physical | 37.93 ± 9.74 | 31.43 ± 8.49 | 4.21 | (<10⁻⁴) * |
| SF12 mental | 52.49 ± 8.57 | 48.46 ± 11.12 | 2.40 | (0.02) |
| BDI | 5.97 ± 4.51 | 11.03 ± 7.76 | −4.69 | (<10⁻⁵) * |
| PANAS positive | 32.28 ± 7.76 | 31.30 ± 7.91 | 0.73 | (0.46) |
| PANAS negative | 16.52 ± 5.11 | 18.59 ± 6.08 | −2.17 | (0.03) |

In CBP+O as compared to CBP−O, all pain and mood measures tended towards or showed worse outcomes, and none indicated improvement. CBP+O and CBP−O patients showed significant differences across multiple emotional, functional and pain properties (two-tailed t-test, *p<0.05 Bonferroni corrected). MPQ=McGill Pain Questionnaire; PCS=Pain Catastrophizing Scale; ODI=Oswestry low back Disability Index; PROMIS=Patient-Reported Outcomes Measurement Information System, subscales are labeled, pain int.=pain interference; SF12=Short Form quality of life scale; BDI=Becks Depression Inventory; PANAS=Positive and Negative Affect Schedule.

TABLE 3

| | CBP (n = 140) | | | CBP − O (n = 70) | | | CBP + O (n = 70) | | |
|---|---|---|---|---|---|---|---|---|---|
| | PC1 | PC2 | PC3 | PC1′ | PC2′ | PC3′ | PC1″ | PC2″ | PC3″ |
| PainDETECT | 0.22 | 0.77 | 0.08 | 0.13 | 0.82 | 0.15 | 0.31 | 0.70 | 0.07 |
| MPQ sensory | 0.16 | 0.88 | −0.01 | 0.05 | 0.91 | −0.05 | 0.23 | 0.81 | 0.05 |
| MPQ affect | −0.01 | 0.82 | 0.15 | −0.07 | 0.70 | 0.09 | 0.01 | 0.88 | 0.17 |
| PCS | 0.28 | 0.42 | 0.53 | 0.44 | 0.23 | 0.59 | 0.17 | 0.61 | 0.47 |
| ODI | 0.73 | 0.43 | 0.21 | 0.76 | 0.32 | 0.14 | 0.62 | 0.51 | 0.26 |
| PROMIS function | −0.86 | −0.07 | −0.17 | −0.84 | 0.19 | −0.04 | −0.83 | −0.23 | −0.21 |
| PROMIS anxiety | 0.21 | 0.01 | 0.74 | 0.34 | −0.01 | 0.64 | 0.18 | 0.08 | 0.78 |
| PROMIS depression | 0.20 | 0.04 | 0.80 | 0.25 | −0.05 | 0.73 | 0.19 | 0.13 | 0.82 |
| PROMIS fatigue | 0.60 | 0.08 | 0.51 | 0.55 | −0.01 | 0.50 | 0.61 | 0.06 | 0.52 |
| PROMIS sleep dis | 0.42 | 0.05 | 0.49 | 0.53 | 0.06 | 0.49 | 0.44 | 0.01 | 0.48 |
| PROMIS social | −0.81 | −0.06 | −0.38 | −0.81 | 0.11 | −0.36 | −0.78 | −0.16 | −0.35 |
| PROMIS pain Int. | 0.82 | 0.08 | 0.32 | 0.85 | −0.11 | 0.26 | 0.74 | 0.17 | 0.32 |
| SF12 physical | −0.88 | −0.18 | −0.04 | −0.88 | −0.14 | −0.03 | −0.84 | −0.18 | −0.01 |
| SF12 mental | −0.05 | −0.26 | −0.79 | 0.07 | 0.03 | −0.81 | −0.08 | −0.34 | −0.78 |
| BDI | 0.39 | 0.23 | 0.68 | 0.25 | 0.03 | 0.77 | 0.41 | 0.20 | 0.70 |
| PANAS positive | −0.18 | 0.15 | −0.61 | −0.16 | 0.43 | −0.43 | −0.19 | −0.02 | −0.68 |
| PANAS negative | 0.11 | 0.45 | 0.67 | −0.01 | 0.36 | 0.70 | 0.20 | 0.45 | 0.67 |
| Eigen value | 4.34 | 2.80 | 4.19 | 4.52 | 2.55 | 3.95 | 4.01 | 3.10 | 4.39 |
| Prob Exp Variance | 0.26 | 0.16 | 0.25 | 0.27 | 0.15 | 0.23 | 0.24 | 0.18 | 0.26 |

Loading of all seventeen measurements on the principal components determined from all patients (PC1, PC2, PC3), CBP−O (PC1′, PC2′, PC3′) and CBP+O (PC1″, PC2″, PC3″). Data from all patients, or separately for CBP+O and CBP−O, mapped to the same three principal components that represent functional disability (PC1), pain quality (PC2), and negative affect (PC3). Group (CBP+O vs. CBP−O) differences for the three PC scores were assessed using analysis of covariance (ANCOVA) with sex, race, age, pain intensity (NRS), pain duration (log), body mass index (BMI), and medication quantification scale (MQS) included as covariates. CBP+O exhibited higher functional disability (PC1) and worse pain quality (PC2) scores compared to CBP−O. The groups had similar negative affect (PC3) (FIG. 1B, Table 4).

TABLE 4

| | PC1 (Functional disability) | | | | PC2 (Pain Quality) | | | | PC3 (Negative affect) | | | |
|---|---|---|---|---|---|---|---|---|---|---|---|---|
| | Beta | ηₚ² | F-value | P-value | Beta | ηₚ² | F-value | P-value | Beta | ηₚ² | F-value | P-value |
| Opioid (CBP − O > | −0.369 | 0.115 | 12.71 | <10⁻³ | −0.305 | 0.078 | 8.29 | <10⁻² | 0.060 | 0.003 | 0.26 | 0.61 |
| CBP + O) | | | | | | | | | | | | |
| Sex (Male > | −0.014 | 0.000 | 0.03 | 0.87 | −0.057 | 0.004 | 0.39 | 0.54 | 0.107 | 0.011 | 1.14 | 0.29 |
| Female) | | | | | | | | | | | | |
| Race (White > | −0.007 | 0.000 | 0.01 | 0.94 | −0.015 | 0.000 | 0.03 | 0.87 | 0.081 | 0.006 | 0.64 | 0.43 |
| Black) | | | | | | | | | | | | |
| Age | 0.187 | 0.039 | 3.93 | 0.06 | −0.064 | 0.004 | 0.41 | 0.56 | −0.236 | 0.048 | 4.99 | 0.03 |
| BMI | 0.247 | 0.068 | 7.16 | 0.01 | −0.007 | 0.000 | 0.00 | 0.94 | −0.124 | 0.014 | 1.42 | 0.24 |
| NRS | 0.148 | 0.026 | 2.62 | 0.11 | 0.372 | 0.140 | 15.90 | <10⁻³ | −0.042 | 0.002 | 0.16 | 0.69 |
| Log Pain duration | −0.118 | 0.017 | 1.65 | 0.20 | 0.006 | 0.000 | 0.00 | 0.95 | −0.002 | 0.000 | 0.00 | 0.99 |
| MQS | 0.070 | 0.005 | 0.46 | 0.50 | −0.033 | 0.001 | 0.10 | 0.75 | 0.089 | 0.006 | 0.60 | 0.44 |

Effect size and significance of opioid use, demographic and pain properties on clinical and behavioral components of CBP patients. Opioid use exacerbated functional disability (PC1) and pain quality (PC2). It was also observed that PCl is worsened with higher BMI, PC2 worsened with higher pain scores, and PC3 improved with age. These additional relationships are consistent with the labels assigned to the three PCs. Significance was determined using ANCOVA. Effect size was computed using partial eta-squared (ηₚ²). ηₚ²<0.05 indicates small effect size; 0.05<ηₚ²B<0.15 indicates medium effect size; ηₚ²>0.15 indicates large effect size indicate small effect size BMI=body mass index; MQS=medication quantification scale. NRS=numerical rating scale.

Even though back pain intensity was matched between CBP−O and CBP+O and PC2 was correlated with back pain intensity in both groups, the consumption of non-opioid medications (MQS) was twice that in CBP+O compared to CBP−O and correlated with back pain intensity only in CBP+O (FIGS. 1G-1H). Overall, the clinical phenotyping indicates that CBP+O 1) report more severe pain qualities, particularly neuropathic-like pain with a larger sensory component; 2) show higher functional disability; 3) do not differ in negative affect from non-opioid users, although their depression ratings were one standard deviation higher than CBP−O; and 4) consume more non-opioid medications than those not on opioids, in proportion with their reported pain intensity. Although opioid consumption was not associated with an improvement in any of the 17 clinical parameters examined, it is essential to note that many of the differences between CBP+O and CBP−O were modest.

Opioid dosage is a strong determinant of medication safety and is considered when deciding to taper or reduce prescribed dose(s) [37]. It was examined how opioid use in CBP+O relates to clinical measures, including withdrawal symptoms and misuse risk. Opioid use was quantified using three measurements:1) the daily prescription converted into morphine milligram equivalent (MME); 2) blood levels of opioids converted to a relative opioid equivalent (ROE, mg/L); and 3) the duration of opioid use (DOU) (FIG. 1C). MME was used to subdivide CBP+O by OUD risk as defined by the Centers for Disease Control and Prevention (CDC) guidelines 38 (FIG. 1C top panel). All three measures—MME, ROE, and DOU—were right-skewed and thus log-transformed. Blood opioids (ROE) in CBP+O reflected their prescription dose (MME) (r=0.48, p<0.001; FIG. 1D-lE scatter plot), but neither blood opioid levels (ROE) nor prescription dose (MME) was strongly associated with the duration of opioid use or with non-opioid medication use (MQS) (FIG. 1D).

Patient withdrawal symptoms and misuse risk was assessed using validated scales (subjective opiate withdrawal scale, SOWS; current opioid misuse measure, COMM). On these scales, 19 CBP+O (27%) showed high misuse risk (COMM>9) and 7 (10%) high withdrawal symptoms (SOWS>20). Scale relationships with back pain intensity, functional disability (PC1), pain quality (PC2), and negative affect (PC3) were investigated. Opioid use characteristics (MME, ROE, and DOU) were not consistently associated with withdrawal or misuse. Only ROE showed a statistically significant positive correlation with functional disability (r=0.47, p<0.01; FIG. 1I). There was no statistically significant association between opioid usage parameters with pain intensity, pain quality, or negative affect scores (PC1-3, FIG. 1F).

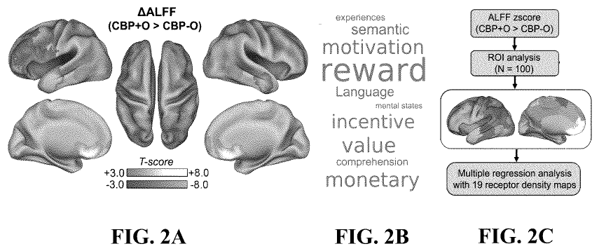

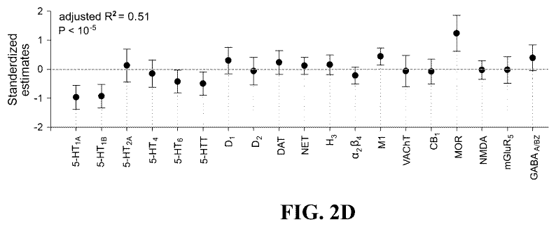

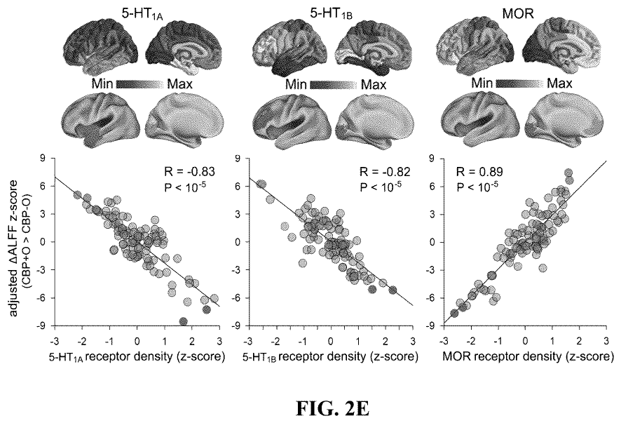

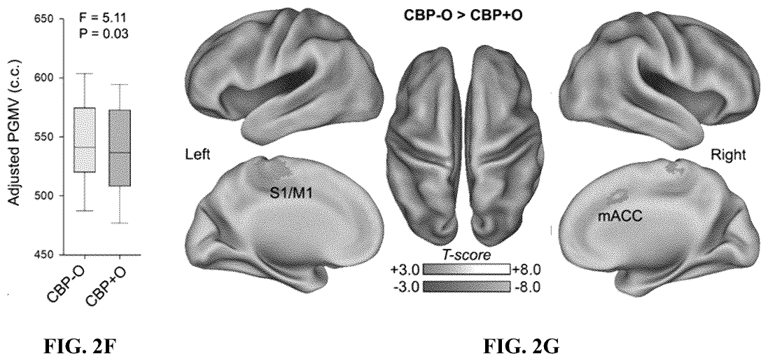

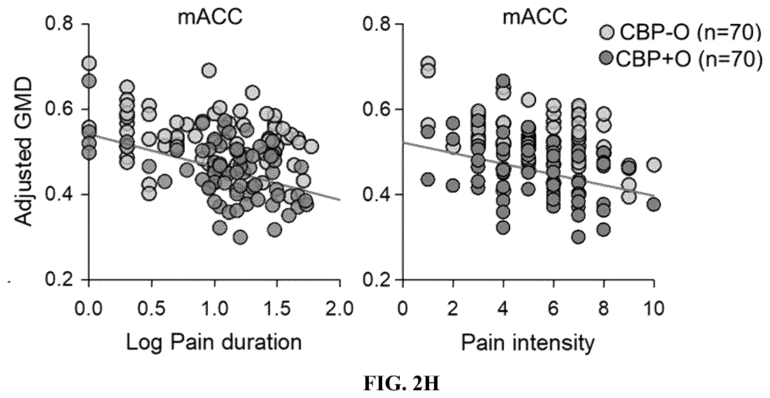

Impact of long-term opioid use on brain structure was examined next. Chronic pain and OUD are each associated with global and local gray matter reorganization [39-42]. Normalized peripheral gray matter volumes (PGMV) were computed for all participants. After adjusting for covariates of no interest (age, sex, race, NRS, pain duration, BMI and MQS), CBP+O showed lower PGMV than CBP−O (p<0.05) (FIG. 2F, Table 5). However, PGMV was not related to opioid use (MME, ROE, and DOU), clinical parameters (PC1-3), or withdrawal and misuse scales (Table 6). Subcortical volumes were also investigated, which did not statistically significantly differ between CBP+O and CBP−O (Table 7).

TABLE 5

| | Peripheral gray matter volume | | | |
|---|---|---|---|---|
| | Beta | ηₚ² | F-value | P-value |
| Opioid (CBP − O > CBP + O) | 0.196 | 0.036 | 5.07 | 0.03 |
| Sex (Male > Female) | −0.264 | 0.084 | 6.64 | 0.01 |
| Race (White > Black) | −0.039 | 0.002 | 0.10 | 0.75 |
| Age (year) | −0.388 | 0.152 | 19.08 | <10⁻⁵ |
| BMI | −0.098 | 0.012 | 1.25 | 0.27 |
| NRS (0-10) | 0.079 | 0.008 | 0.63 | 0.43 |
| Log Pain duration (year) | 0.140 | 0.024 | 2.24 | 0.14 |
| MQS | −0.057 | 0.003 | 0.32 | 0.57 |

Table 5 shows the analysis of covariance for peripheral gray matter volume with opioid use (CBP−O>CBP+O), demographics (sex, race, age, BMI), and pain (NRS, Log Pain Duration) parameters. Effect size and significance of opioid use, demographic and pain properties on peripheral gray matter volume. Significance was determined using ANCOVA. Effect size was computed using partial eta-squared (ηₚ²). Long-term opioid use, older age, and females exhibited smaller gray matter volume. Relative to the effect size observed for one year of aging, an average of 6 years of opioid consumption decreases gray matter volume by a magnitude equivalent to about 0.5 years of aging. ηₚ²<0.05 indicates small effect size; 0.05<ηₚ²B<0.15 indicates medium effect size; ηₚ²>0.15 indicates large effect size. BMI=body mass index; MQS=medication quantification scale. NRS=numerical rating scale.

TABLE 6

| | Peripheral gray matter volume | | | |
|---|---|---|---|---|
| | Beta | ηₚ² | F-value | P-value |
| Log MME | 0.12 | 0.01 | 0.34 | 0.56 |
| Log ROE | −0.18 | 0.03 | 0.80 | 0.38 |
| Log DOU | 0.20 | 0.05 | 1.50 | 0.23 |
| SOWS | −0.31 | 0.07 | 2.21 | 0.15 |
| COMM | −0.28 | 0.06 | 2.07 | 0.16 |
| PC1- Functional disability | 0.02 | 0.00 | 0.02 | 0.88 |
| PC2 - Pain quality | 0.12 | 0.02 | 0.49 | 0.49 |
| PC3 - Negative affect | 0.12 | 0.01 | 0.34 | 0.56 |

As shown in Table 6, peripheral gray matter volume in CBP+O (n=70) does not depend on opioid consumption (MME), blood levels of opioids (ROE), duration of opioid use (DOU), signs of withdrawal (SOWS) or misuse (COMM), and clinical parameters (PC1-3). Relationship between PGMV and clinical parameters were determined using a linear regression model. There were no significant associations between PGMV and clinical parameters. These results suggests that either gray matter volume is an indication of opioid use vulnerability or that it is a consequence of long-term adaptations that have become independent of clinical signs. Effect size was computed using partial eta-squared (ηₚ²). ηₚ²<0.05 indicates small effect size; 0.05ηₚ²<0.15 indicates medium effect size; ηp2>0.15 indicates large effect size indicates small effect size. MME=Morphine Milligram Equivalents; ROE=Relative Opioid Equivalent; DOU=duration of opioid use; SOWS=subjective opioid withdrawal scale; COMM=current opioid misuse measure.

TABLE 7

| | Opioid | Sex | Race | | | | Log | |
|---|---|---|---|---|---|---|---|---|
| | (CBP − O > | (Females > | (Black > | Age | | Pain | Pain | |
| | CBP + O) | Male) | White) | (years) | BMI | Intensity | duration | MQS |
| Right Thalamus | 3.78 | 5.46 | 0.58 | 15.21 | 0.81 | 0.84 | 0.40 | 3.43 |
| | −0.20 | −0.21 | −0.07 | −0.37 | −0.08 | −0.08 | 0.05 | −0.19 |
| | 0.03 | 0.05 | 0.01 | 0.13 | 0.01 | 0.01 | 0.01 | 0.03 |
| Left Thalamus | 1.83 | 5.46 | 0.26 | 24.19 | 1.34 | 3.25 | 0.12 | 1.15 |
| | −0.13 | 0.20 | 0.05 | −0.45 | −0.10 | −0.16 | 0.03 | −0.10 |
| | 0.02 | 0.05 | 0.00 | 0.19 | 0.01 | 0.03 | 0.00 | 0.01 |
| Right Caudate | 0.00 | 7.60 | 0.09 | 4.48 | 2.04 | 0.00 | 0.71 | 0.04 |
| | 0.00 | 0.27 | 0.03 | −0.22 | −0.14 | 0.00 | −0.08 | −.02 |
| | 0.00 | 0.07 | 0.00 | 0.04 | 0.02 | 0.00 | 0.01 | 0.00 |
| Left Caudate | 1.27 | 4.50 | 0.22 | 6.71 | 1.79 | 0.81 | 0.10 | 0.09 |
| | −0.12 | 0.18 | 0.04 | −0.28 | −0.12 | −0.08 | 0.02 | −0.09 |
| | 0.02 | 0.04 | 0.00 | 0.23 | 0.01 | 0.01 | 0.00 | 0.00 |
| Right Putamen | 0.99 | 20.79 | 0.14 | 16.72 | 0.58 | 0.61 | 0.29 | 3.05 |
| | −0.11 | 0.33 | −0.07 | −0.36 | 0.04 | −0.07 | 0.06 | −0.18 |
| | 0.01 | 0.11 | 0.00 | 0.14 | 0.00 | 0.01 | 0.00 | 0.03 |
| Left Putamen | 0.44 | 20.65 | 0.37 | 42.47 | 0.03 | 3.42 | 0.16 | 0.04 |
| | −0.06 | 0.37 | −0.05 | −0.56 | 0.01 | −0.15 | 0.03 | 0.00 |
| | 0.00 | 0.18 | 0.00 | 0.30 | 0.00 | 0.03 | 0.00 | 0.00 |
| Right Pallidum | 1.40 | 1.55 | 0.06 | 7.05 | 1.58 | 0.07 | 0.06 | 8.78 |
| | −0.12 | 0.11 | −0.02 | −0.26 | 0.12 | −0.02 | −0.02 | −0.31 |
| | 0.01 | 0.01 | 0.00 | 0.07 | 0.02 | 0.00 | 0.00 | 0.08 |
| Left Pallidum | 0.03 | 5.72 | 0.18 | 3.06 | 5.35 | 0.00 | 0.08 | 5.46 |
| | 0.00 | 0.25 | −0.09 | −0.18 | 0.21 | 0.00 | −0.02 | −0.21 |
| | 0.00 | 0.06 | 0.01 | 0.03 | 0.05 | 0.00 | 0.00 | 0.03 |
| Right | 0.36 | 0.93 | 0.32 | 7.07 | 0.40 | 6.88 | 1.17 | 1.64 |
| Hippocampus | −0.07 | −0.09 | −0.06 | −0.27 | −0.06 | −0.25 | −0.11 | −0.14 |
| | 0.00 | 0.01 | 0.00 | 0.07 | 0.00 | 0.07 | 0.01 | 0.02 |
| Left | 0.95 | 1.71 | 0.07 | 4.40 | 0.00 | 3.29 | 0.21 | 0.36 |
| Hippocampus | −0.08 | −0.13 | 0.00 | −0.19 | 0.00 | −0.20 | −0.04 | −0.05 |
| | 0.00 | 0.02 | 0.00 | 0.04 | 0.00 | 0.04 | 0.00 | 0.00 |
| Right Amygdala | 0.47 | 0.01 | 6.78 | 0.11 | 0.90 | 0.00 | 2.99 | 0.16 |
| | 0.07 | 0.00 | −0.26 | −0.03 | 0.09 | 0.00 | −0.17 | −0.04 |
| | 0.00 | 0.00 | 0.65 | 0.00 | 0.01 | 0.00 | 0.03 | 0.00 |
| Left Amygdala | 1.50 | 0.20 | 2.47 | 3.24 | 1.64 | 2.57 | 0.00 | 1.97 |
| | 0.13 | −0.04 | −0.16 | −0.17 | 0.14 | −0.17 | 0.00 | −0.15 |
| | 0.02 | 0.00 | 0.04 | 0.03 | 0.02 | 0.04 | 0.00 | 0.03 |
| Right NAc | 3.02 | 0.53 | 0.07 | 35.59 | 0.11 | 3.61 | 0.00 | 0.51 |
| | −0.17 | −0.06 | −0.02 | −0.53 | −0.02 | −0.18 | 0.00 | −0.07 |
| | 0.03 | 0.01 | 0.00 | 0.27 | 0.00 | 0.05 | 0.00 | 0.00 |
| Left NAc | 0.33 | 1.42 | 0.08 | 51.88 | 0.02 | 1.72 | 0.43 | 5.21 |
| | −0.05 | 0.12 | −0.08 | −0.60 | 0.00 | −0.15 | 0.06 | −0.20 |
| | 0.00 | 0.02 | 0.01 | 0.31 | 0.00 | 0.02 | 0.00 | 0.03 |

Table 7 shows the analysis of covariance for subcortical volumes with opioid use (CBP−O>CBP+O), demographics (sex, race, age, BMI), and pain parameters (intensity, duration, MQS). Data presented as F-value/Beta/effect size (ηₚ²). Shaded cells represent significant effects (p<0.05). Subcortical regions showed no significant differences in volume between CBP+O and CBP−O patients. The important outcome is the lack of subcortical volume differences between CBP+O and CBP−O. Thalamus, caudate, and putamen showed lower volumes in females compared to males. Right hippocampus volume was negatively associated with pain intensity, while putamen volumes were negatively related to non-opioid medication use. The right amygdala showed lower volume in White compared to Black. Most regions exhibited decreased volume in relationship to age. ηₚ²<0.05 indicates small effect size; 0.05<ηₚ²B<0.15 indicates medium effect size; ηₚ²>0.15 indicates large effect size. BMI=body mass index; MQS=medication quantification scale; NAc=nucleus accumbens.

Whole-brain voxel-based morphometry (VBM) was used to investigate regional grey matter density (GMD) differences between CBP+O and CBP−O. After adjusting for covariates of no interest, two clusters were found localized to (1) the left primary sensorimotor cortex (Si/Mi) and (2) the mid-anterior cingulate cortex (mACC), which showed decreased GMD in CBP+O. The S1/M1 cluster was associated with sensorimotor function, while mACC with pain, nociception, and arousal, using Neurosynth reverse inference decoder (FIG. 2G, Table 8). In addition, mACC GMD was negatively correlated with both backpain intensity (p=0.02) and duration (p=0.01) in both groups (FIG. 2H, Table 9). Similar to our PGMV analysis, localized GMD changes in mACC and S1/M1 were not statistically significantly associated with opioid use, withdrawal, misuse, or clinical parameters (PC1-3) in CBP+O patients (Table 10). Overall, whole-brain gray matter decreases were modest, and focal decreases in cortical gray matter volume were associated with long-term opioid use in CBP+O.

TABLE 8

| Coordinates (mm) | | | Size | | |
|---|---|---|---|---|---|
| x | y | z | (voxels) | T-score | Associated terms (Neurosynth reverse inference) |

CBP − O > CBP + O

| | | | | | | |
|---|---|---|---|---|---|---|
| mACC | 10 | 8 | 38 | 80 | 4.78 | Autonomic control, aversive, pain, noxious, arousal |
| Left S1/M1 | −10 | −32 | 62 | 503 | 5.84 | Sensorimotor, motor function, movement, motor, |
| | | | | | | motor imagery |

Table 8 shows regional decreases in gray matter density in patients on long-term opioid use. Brain regions that showed significantly decreased gray matter density in CBP+O compared to CPB−O. Terms associated with brain regions were determined using reverse inference from Neurosynth. Coordinates in MNI space; mACC=middle anterior cingulate cortex; S1/M1=primary sensorimotor cortex.

TABLE 9

| | mACC | | | | S1/M1 | | | |
|---|---|---|---|---|---|---|---|---|
| | Beta | ηₚ² | F-value | P-value | Beta | ηₚ² | F-value | P-value |
| Opioid (CBP − O > CBP + O) | 0.572 | 0.270 | 36.25 | <10⁻⁶ | 0.562 | 0.254 | 33.31 | <10⁻⁶ |
| Sex (Male > Female) | −0.039 | 0.002 | 0.23 | 0.63 | −0.137 | 0.026 | 2.66 | 0.11 |
| Race (White > Black) | 0.190 | 0.050 | 5.18 | 0.03 | −0.097 | 0.013 | 1.28 | 0.26 |
| Age | −0.144 | 0.028 | 2.78 | 0.10 | −0.057 | 0.004 | 0.42 | 0.52 |
| BMI | −0.108 | 0.016 | 1.63 | 0.20 | −0.055 | 0.004 | 0.39 | 0.53 |
| NRS | −0.196 | 0.053 | 5.51 | 0.02 | 0.068 | 0.006 | 0.63 | 0.43 |
| Log Pain duration | −0.228 | 0.069 | 7.30 | 0.01 | 0.042 | 0.002 | 0.24 | 0.62 |
| MQS | 0.008 | 0.000 | 0.01 | 0.94 | 0.060 | 0.004 | 0.39 | 0.54 |

Table 9 shows the analysis of covariance for cortical gray matter density changes (mACC, S1/M1) in patients on long-term opioid use with opioid use (CBP−O>CBP+O), demographics (sex, race, age, BMI), and pain (NRS, Pain duration, MQS) parameters. Opioid use, race, pain intensity (NRS), and pain duration were correlated with mACC gray matter density decrease. Only opioid use was correlated with S1/M1 gray matter density decrease. Significance was determined using ANCOVA. Effect size was computed using partial eta-squared (ηₚ²). ηₚ²<0.05 indicates small effect size; 0.05<ηₚ²<0.15 indicates medium effect size; ηₚ²>0.15 indicates large effect size; BMI=body mass index; MQS=medication quantification scale. NRS=numerical rating scale. mACC=middle anterior cingulate cortex. S1/M1=primary sensorimotor cortex.

TABLE 10

| | mACC | | | | S1/M1 | | | |
|---|---|---|---|---|---|---|---|---|
| | Beta | ηₚ² | F-value | P-value | Beta | ηₚ² | F-value | P-value |
| Log MME | 0.13 | 0.01 | 0.39 | 0.54 | 0.04 | 0.00 | 0.04 | 0.85 |
| Log ROE | −0.23 | 0.04 | 1.23 | 0.28 | 0.14 | 0.01 | 0.40 | 0.53 |
| Log DOU | 0.06 | 0.00 | 0.15 | 0.70 | −0.05 | 0.00 | 0.08 | 0.78 |
| SOWS | 0.02 | 0.00 | 0.01 | 0.93 | 0.16 | 0.02 | 0.51 | 0.48 |
| COMM | 0.05 | 0.00 | 0.07 | 0.79 | −0.07 | 0.00 | 0.12 | 0.73 |
| PC1- Functional disability | −0.09 | 0.01 | 0.32 | 0.58 | 0.01 | 0.00 | 0.01 | 0.94 |
| PC2 - Pain quality | 0.12 | 0.02 | 0.49 | 0.49 | −0.19 | 0.03 | 1.05 | 0.31 |
| PC3 - Negative affect | −0.31 | 0.08 | 2.59 | 0.12 | 0.05 | 0.00 | 0.07 | 0.80 |

Table 10 shows no relationship between gray matter density for regions decreased in CBP+O (mACC, Si/M1) with opioid use measures (MME, ROE, DOU), clinical parameters (SOWS, COMM), and principal components (PC1-3) in CBP+O patients. Effect size was computed using partial eta-squared (ηₚ²). ηₚ²<0.05 indicates small effect size; 0.05<ηₚ²<0.15 indicates medium effect size; ηₚ²>0.15 indicates large effect size. All regressions were performed after correcting for age, sex, race, BMI, and MQS effects. MME=Morphine Milligram Equivalents; ROE=Relative Opioid Equivalent; DOU=duration of opioid use; SOWS=subjective opioid withdrawal scale; COMM=current opioid misuse measure. To examine the impact of opioid exposure on the white matter, a subsample of the subjects, 58 CBP+O was contrasted and matched 58 CBP−O, white matter properties using a whole-brain skeletal fractional anisotropy (FA) contrast, which did not yield any statistically significant differences between the two groups (data not shown).

A primary hypothesis explored was that the groups will differ in ongoing brain activity. Resting state fMRI signal was used to test this hypothesis. The power spectrum of brain activity signals is related to various brain functional properties [43,44]. Resting-state fMRI was used to localize voxel-wise differences in the amplitude of low-frequency fluctuations (ALFF, which reflects the low-frequency energy of spontaneous neural activity [45]) between CBP+O and CBP−O while adjusting for age, sex, pain intensity, pain duration, BMI, MQS, and head motion (AALFF, CBP+O>CBP−O). Voxel-wise corrections was also included for scanner signal-to-noise ratios and GMD. Compared to CBP−O, CBP+O showed greater ALFF in five distinct clusters, the largest of which (5,050 voxels) encompassed multiple MCL structures, including bilateral nucleus accumbens (NAc), amygdala, subgenual cingulate, medial/orbital prefrontal cortex, hippocampus, and brain stem. Other clusters in which CBP+O had greater ALFF included the lateral occipital cortex, the middle prefrontal cortex, and the right and left posterior portions of the inferior temporal gyrus. CBP+O patients had lower ALFF in 3 clusters, including the left dorsolateral prefrontal cortex (dlPFC, 3,955 voxels) and the right and left anterior part of the mid-temporal gyrus (FIG. 2A, Table 11). Greater ALFF in CBP+O was localized to brain regions involved in reward, motivation, incentive, and value processing, while lower ALFF in CBP+O was localized to brain regions involved in language and mental states (FIG. 2b). Similar to the morphological results, localized ALFF changes were not statistically significantly associated with opioid use, withdrawal, misuse, or clinical parameters (PC1-3) in CBP+O patients (Table 12).

TABLE 11

| Coordinates (mm) | | | Size | | |
|---|---|---|---|---|---|
| x | y | z | (voxels) | T-score | Associated terms (Neurosynth reverse inference) |

Regions that exhibited increased ALFF in CBP + O (CBP + O > CBP − O)

| | | | | | | |
|---|---|---|---|---|---|---|
| right pITG | 56 | −50 | −8 | 325 | 4.01 | Expression, facial, response time, perception, threating |
| left pITG | −54 | −50 | −14 | 410 | 4.22 | Words, form, visual words, subsequent memory, orthographic |
| mPFC | 4 | 60 | 2 | 823 | 6.01 | Autobiographical, default-mode, cognitive, intention |
| right LOC | 26 | −64 | 52 | 910 | 5.45 | Attention, spatial, calculation, rotation, orientation |
| MCL | 2 | 20 | −10 | 5050 | 7.89 | Reward, value, motivation, monetary, incentive |
| right NAC | 8 | 10 | −8 | 164 | 7.13 | |
| left NAc | −10 | 10 | −10 | 162 | 7.05 | |
| sgACC | 2 | 24 | −14 | 516 | 6.85 | |
| aACC | −2 | 34 | −4 | 404 | 6.11 | |
| right Amyg | 18 | −2 | −18 | 44 | 4.01 | |
| left Amyg | −20 | −2 | −20 | 28 | 3.79 | |
| Brainstem | −2 | −26 | −8 | 86 | 3.25 | |

Regions that exhibited decreased ALFF in CBP + O (CBP + O < CBP − O)

| | | | | | | |
|---|---|---|---|---|---|---|
| right aMTG | 54 | −8 | −20 | 1275 | −6.58 | Mind, mental states, theory, social, experiences |
| left aMTG | −54 | −8 | −22 | 1309 | −6.94 | Semantic, sentences, mentalizing, linguistic, theory of mind |
| left dlPFC | −46 | 24 | 22 | 3955 | −5.08 | Language, phonological, syntactic, verb, demands |
| left IFG | −50 | 15 | 6 | 1478 | −5.06 | |
| left MFG | −42 | 30 | 32 | 2040 | −5.02 | |

Table 11 shows regional ALFF changes in patients on long-term opioid use. The table shows Coordinates in mm (MNI space), cluster size and T-score of brain regions that showed significant differences in ALFF between CBP+O and CBP−O (Threshold free Cluster corrected, T-score >2.3, p<0.01). The local coordinates, sizes, and T-scores for distinct subregions within the MCL and dlPFC clusters are shown. The top 5 associated terms with any given cluster were determined using reverse inference from Neurosynth. ALFF=Amplitude of low frequency fluctuations; pITG=posterior inferior temporal gyrus; MCL=meso-cortical limbic. mPFC=middle prefrontal cortex; LOC=Lateral occipital cortex; aMTG=anterior middle temporal gyrus; dlPFC=dorsolateral prefrontal cortex. Amyg=amygdala; NAc=Nucleus accumbens; sgACC=subgenual division of anterior cingulate cortex; aACC=anterior division of division of anterior cingulate cortex; IFG=Inferior frontal gyrus; MFG=Middle frontal gyrus.

TABLE 12

| | | | | | | | |
|---|---|---|---|---|---|---|---|
| Log MME | Log ROE | Log DOU | SOWS | COMM | PC1 | PC2 | PC3 |
| R-value | R-value | R-value | R-value | R-value | R-value | R-value | R-value |
| (P-value) | (P-value) | (P-value) | (P-value) | (P-value) | (P-value) | (P-value) | (P-value) |

Regions that exhibited increased ALFF (CBP + O > CBP − O)

| | | | | | | | | |
|---|---|---|---|---|---|---|---|---|
| right pITG | 0.13 (0.33) | −0.05 (0.69) | −0.17 (0.19) | 0.08 (0.52) | −0.04 (0.76) | 0.10 (0.40) | −0.02 (0.85) | 0.12 (0.37) |
| left pITG | 0.04 (0.76) | 0.03 (0.79) | −0.15 (0.26) | −0.03 (0.79) | 0.11 (0.40) | 0.20 (0.10) | 0.06 (0.64) | 0.11 (0.39) |
| mPFC | −0.05 (0.67) | −0.07 (0.60) | −0.09 (0.50) | 0.04 (0.76) | 0.08 (0.52) | 0.08 (0.52) | 0.08 (0.53) | 0.15 (0.26) |
| right LOC | 0.21 (0.09) | 0.22 (0.08) | −0.05 (0.68) | 0.11 (0.39) | 0.13 (0.35) | 0.10 (0.41) | 0.10 (0.40) | 0.01 (0.93) |
| MCL | 0.12 (0.38) | 0.01 (0.97) | −0.04 (0.74) | 0.10 (0.40) | −0.04 (0.76) | 0.16 (0.27) | 0.05 (0.68) | 0.14 (0.29) |

Regions that exhibited decreased ALFF (CBP + O < CBP − O)

| | | | | | | | | |
|---|---|---|---|---|---|---|---|---|
| right aMTG | −0.15 (0.28) | −0.03 (0.81) | 0.02 (0.89) | 0.14 (0.30) | −0.02 (0.86) | −0.04 (0.76) | 0.01 (0.94) | 0.02 (0.86) |
| left aMTG | −0.02 (0.86) | 0.08 (0.52) | −0.01 (0.93) | 0.07 (0.59) | 0.07 (0.59) | −0.07 (0.59) | 0.06 (0.69) | 0.06 (0.69) |
| left dlPFC | −0.22 (0.08) | −0.02 (0.88) | −0.01 (0.93) | −0.04 (0.74) | 0.02 (0.87) | −0.13 (0.33) | −0.19 (0.10) | 0.04 (0.76) |

Table 12 shows brain regional activity (ALFF) changes between CBP+O and CBP−O were not correlated with opioid use (MME, ROE, DOU), clinical parameters (SOWS, COMM), and characteristics of back pain (PC1-3), in CBP+O patients. Data show the correlation of ALFF with MME, ROE, DOU, COMM, SOWS, PCi-functional disability, PC2-pain quality, and PC3-negative affect of for regions that showed significant ALFF differences between CBP+O and CBP−O. ALFF values were corrected for age, sex, race, pain intensity, BMI and MQS prior to correlation. ALFF=Amplitude of low-frequency fluctuations; pITG=posterior inferior temporal gyrus; MCL=meso-cortical limbic; mPFC=middle prefrontal cortex; LOC=Lateral occipital cortex; aMTG=anterior middle temporal gyrus; dlPFC=dorsolateral prefrontal cortex. MME=Morphine Milligram Equivalents; ROE=Relative Opioid Equivalent; DOU=duration of opioid use; SOWS=subjective opioid withdrawal scale; COMM=current opioid misuse measure.

Given that the CBP+O cyclically and daily perturb their brain molecular properties by regularly ingesting opioids and also our recent evidence shows that pain perception is better conceptualized as a whole-brain process 30, a new methodology was developed to relate the brain activity of regular opioid use to cortical receptor distribution-dependent activity. It was studied how receptor density distributions mapped onto CBP+O vs. CBP−O activity differences. Specifically, how much of the difference in brain activity (AALFF between CBP+O and CBP−O) can be explained by neurotransmitter receptor densities? To address this issue, multiple regression was used to model ΔALFF using 19 neurotransmitter receptor density maps when the cortex is divided into 100 regions (constructed by amalgamating PET images from 1,238 healthy participants across nine neurotransmitter systems [31]) (FIG. 2C). The receptor density maps explained 51% of the variance of ΔALFF (p<0.01) (FIG. 2D). The serotonin (5-HT_{1A} and 5-HT_{1B}) receptors and the μ-opioid (MOR) receptor accounted for the largest explanatory variance (ΔR² for 5-HT_{1A} receptor map was 19.6%, for 5-HT_{1B} it was 17.6%, and for MOR it was 13.7%; together their ΔR² was 27.4%). After adjusting for the remaining receptors, the 5-HT_{1A} and 5-HT_{1B} receptor distributions in the cortex were strongly negatively correlated with ΔALFF, while the MOR receptor cortical distribution was strongly positively correlated with the ΔALFF (FIG. 2E). The strength of these correlations highlights that their influence on ΔALFF follows an almost uniform proportionality throughout the cortex (e.g., the 5-HT_{1A} receptor expression anywhere in the cortex accounts for a decrease of ~⅔ AU in local ALFF).

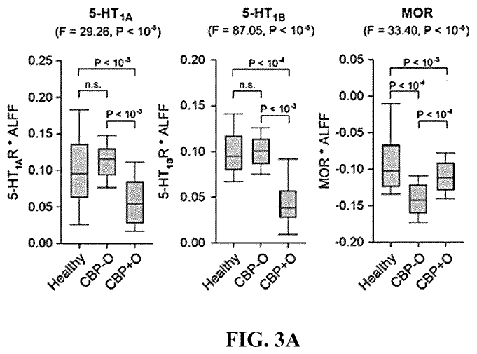

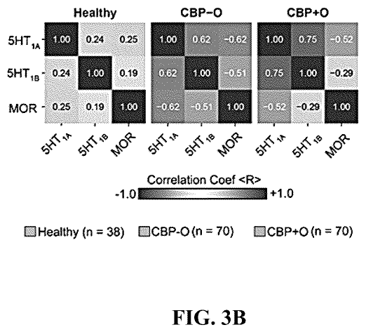

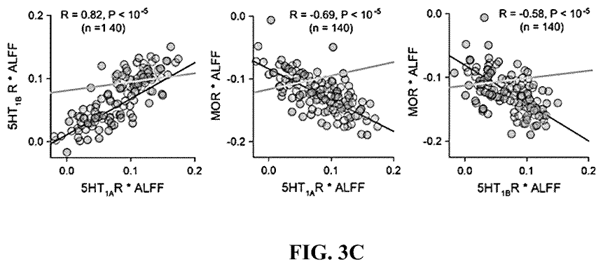

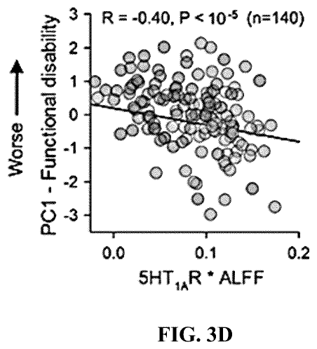

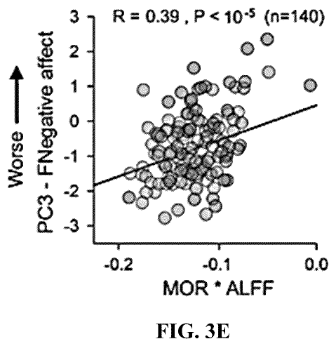

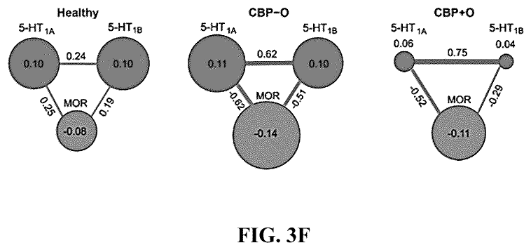

The spatial uniformity of the relationship between ΔALFF and all three receptor density maps enables the derivation of a unitary measure of receptor-related activity across the cortex. This measure was used to test for group differences and to examine relationships with clinical characteristics. Cortex-wide receptor-related activity for each subject was computed as the normalized dot product between regional ALFF and the corresponding receptor density distribution. This resulted in one value per subject representing cortical receptor-related activity, which was designated as 5-HT_{1A}R*ALFF, 5-HT_{1B}R*ALFF, and MOR*ALFF. Overall, across healthy subjects and CBP−O and CBP+O, the 5-HT_{1A}R*ALFF and 5-HT_{1B}R*ALFF were positive (enhancing brain activity, or excitatory), while the MOR-specific activity (MOR*ALFF) was negative (decreasing brain activity, or inhibitory). Both serotonin-related activities were less in CBP+O than in CBP−O and healthy controls, while MOR-related activity was higher (less inhibition) in CBP+O (FIG. 3B). 5-HT_{1A}R*ALFF and 5-HT_{1B}R*ALFF were positively correlated with each other and negatively with MOR*ALFF in both patient groups, but not in the healthy (FIG. 3C).

The relationship between receptor-related activity and clinical parameters was examined using multiple regression analysis. Across all 140 CBP−O and CBP+O patients, 5-HT_{1A}R*ALFF was negatively related to the PC1-functional disability (FIG. 3D), while MOR*ALFF was positively related to the PC3-negative affect (FIG. 3E). PC2-pain quality was not related to any of the three receptor-related activities. These results can be summarized in a model with three nodes (FIG. 3F), which shows that long-term opioid use predominantly impacts serotoninergic activity and partially renormalizes opioidergic activity.

The results disclosed herein demonstrate a strong relationship between the three receptor-related activities and the states of CBP−O and CBP+O. To establish a causal relationship concerning how these receptors interact, their response to various opioid conditions and perturbations were assessed. 5-HT_{1A}, 5-HT_{1B}, and MOR-related brain activity was investigated in 4 different conditions: (1) high vs. low opioid consumption, (2) before and after brief abstinence from opioid use, and (3-4) responses to a non-pharmacological, multi-disciplinary intervention leading to either successful (3) or unsuccessful (4) tapering.

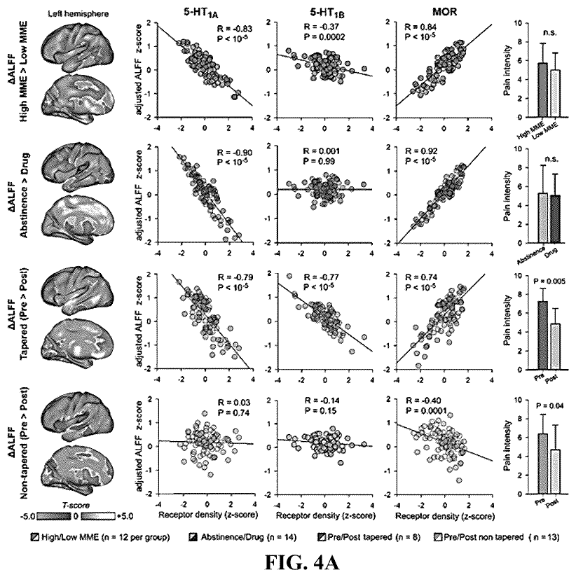

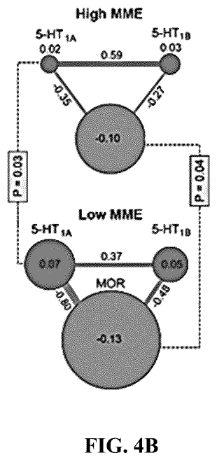

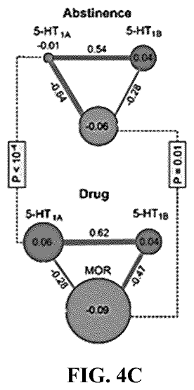

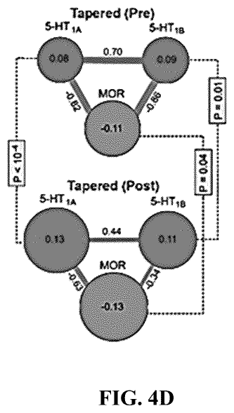

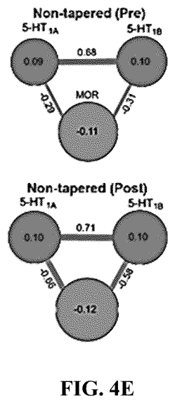

High vs. low opioid consumption: The effect of opioid consumption dose was determined by computing ΔALFF between high opioid (n=12, MME>50) and low opioid (n=12, MME<20) CBP+O patients matched for age, sex, pain intensity, and duration as well as opioid use duration (Table 13). Localized regions that showed ALFF changes with long-term opioid use (CBP+O>CBP−O) did not statistically significantly differ between high and low opioid users (Table 14). Despite the paucity of salient local differences, ΔALFF strongly negatively related to 5-HT_{1A} and 5-HT_{1B} and positively correlated with the MOR density map (FIG. 4A, first row). In addition, patients with high doses of opioids showed lower 5-HT_{1A}R*ALFF and higher MOR*ALFF than patients on low doses of opioids (FIG. 4B). A pattern that closely recapitulates what was observed in the overall group of CBP+O in comparison to CBP−O (FIG. 3F).

TABLE 13

| | | |
|---|---|---|
| High MME | Low MME | |
| (n = 12) | (n = 12) | |
| mean ± s.d. | mean ± s.d. | p-value |

Demographic and general health characteristics

| | | | |
|---|---|---|---|
| Age (years) | 56.58 ± 11.39 | 58.00 ± 7.15 | 0.72 |
| Sex/female (%) | 4 (33.33%) | 4 (33.33%) | 1 |
| BMI | 29.35 ± 6.42 | 31.05 ± 5.91 | 0.54 |

Pain characteristics

| | | | |
|---|---|---|---|
| Pain intensity (NRS) | 5.75 ± 2.09 | 5.00 ± 1.81 | 0.45 |
| Pain duration (years) | 14.33 ± 8.47 | 19.67 ± 14.67 | 0.29 |

Opioid consumption and behavior

| | | | |
|---|---|---|---|
| MME | 136.14 ± 70.29 | 14.69 ± 9.54 | <10⁻³ |
| ROE (mg/L) | 0.014 ± 0.02 | 0.0017 ± 0.004 | <10⁻² |
| Duration of opioid use (years) | 8.50 ± 6.59 | 8.42 ± 7.84 | 0.98 |
| SOWS | 15.17 ± 11.15 | 8.83 ± 10.74 | 0.17 |
| COMM | 11.00 ± 6.64 | 6.00 ± 4.89 | 0.06 |

Table 13 shows patient demographics and general health for high and low MME groups. Patients on high opioids (n=12) and low opioid (n=12) were matched one-to-one for age, sex, and BMI (two-tailed unpaired t-tests or chi-square tests were performed). They were also matched for pain intensity and duration. Opioid consumption was quantified using the daily prescription converted into MME, the blood levels of opioids converted to an ROE (mg/L), and the duration of opioid use in years. On average opioid daily consumption and blood concentrations were 10 times lower in patients on low opioids compared to those on high opioids. Both groups had similar duration of opioid use. Patients on high opioids showed slightly higher withdrawal symptoms (SOWS) and misuse (COMM). BMI=Body Mass Index; MME=Morphine Milligram Equivalents; ROE=Relative Opioid Equivalent; DOU=duration of opioid use; SOWS=subjective opioid withdrawal scale; COMM=current opioid misuse measure.

TABLE 14

| | | | |
|---|---|---|---|
| CBP + O | CBP + O | | |
| (MME >=50) | (MME <50) | | |
| (mean ± s.d.) | (mean ± s.d.) | T-value | p-value |

Regions that exhibited increased ALFF (CBP + O > CBP − O)

| | | | | |
|---|---|---|---|---|
| right pITG | 1.04 ± 0.02 | 1.03 ± 0.02 | 0.32 | 0.752 |
| left pITG | 1.07 ± 0.03 | 1.07 ± 0.03 | −0.17 | 0.867 |
| mPFC | 0.98 ± 0.02 | 1.00 ± 0.02 | −1.46 | 0.146 |
| right LOC | 1.04 ± 0.03 | 1.03 ± 0.02 | 1.29 | 0.199 |
| MCL | 1.03 ± 0.02 | 1.04 ± 0.03 | −1.02 | 0.318 |

Regions that exhibited decreased ALFF (CBP + O < CBP − O)

| | | | | |
|---|---|---|---|---|
| right aMTG | 0.99 ± 0.02 | 0.98 ± 0.02 | 0.05 | 0.996 |
| left aMTG | 0.98 ± 0.02 | 0.97 ± 0.01 | 1.33 | 0.187 |
| left dlPFC | 0.97 ± 0.02 | 0.96 ± 0.02 | 0.763 | 0.448 |

Table 14 shows regional activity (ALFF) changes in CBP+O did not differ between low (MME<50) and high (MME>50) opioid consumption. Data show differences in ALFF ROI between CBP+O patients with high MME (MME>=50) and those with low MME (MME<50). There were no differences between groups for all regions examined. ALFF=Amplitude of low-frequency fluctuations; pITG=posterior inferior temporal gyrus; mPFC=middle prefrontal cortex; LOC=Lateral occipital cortex; aMTG=anterior middle temporal gyrus; dlPFC=dorsolateral prefrontal cortex. MME=Morphine Milligram Equivalents; ROE=Relative Opioid Equivalents; BMI=body mass index; MQS=medication quantification scale.

Brief opioid abstinence: In a subsample of 14 CBP+O patients, their stable opioid status was perturbed by requesting the participants who were taking short-acting opioids only to briefly refrain from taking their opioids overnight (19.4±6.7 hours) and undergo a second brain resting state scan. Blood samples confirmed patients' abstinence, as there were no or minimal opioids detected in the blood, especially in comparison to their first scan, which was collected within 3.01±2.75 hours of opioid consumption (Table 15). Pain intensity did not differ between baseline and abstinence, but signs of withdrawal increased with abstinence (Table 15). Most brain regions that showed significant ALFF differences between CBP+O and CBP−O showed minimal changes following opioid abstinence. Only the left pITG showed decreased ALFF, while the right aMTG showed increased ALFF following abstinence (Table 16). The within-subject ΔALFF between before and after abstinence was strongly negatively associated with 5-HT1A receptor distribution and positively associated with the MOR receptor distribution in the cortex (FIG. 4A, second row). In addition, opioid abstinence was associated with a decrease in 5-HT_{1A}R*ALFF and an increase in MOR*ALFF, but no changes in 5-HT_{1B}R*ALFF (FIG. 4C). This result sheds light on the temporal dynamics of opioid exposure's effects on the brain, suggesting that 5-HT_{1B} receptor-related activity is a consequence of long-duration adaptations.

TABLE 15

| | Baseline | Abstinence | | |
|---|---|---|---|---|
| | (mean ± s.e.m.) | (mean ± s.e.m) | T-value | p-value |
| Log ROE | −3.42 ± 0.51 | −4.51 ± 0.29 | 4.57 | <0.001 |
| Pain intensity | 5.07 ± 0.51 | 5.28 ± 0.79 | −0.42 | 0.68 |
| (NRS) | | | | |
| SOWS | 6.14 ± 1.22 | 7.57 ± 0.03 | −2.16 | <0.05 |

Table 15 shows behavioral changes between baseline and following a brief period of opioid abstinence in 14 patients on long-term opioids. Blood levels of opioids (Log ROE) were significantly lower after opioid abstinence. Opioid abstinence results in increased withdrawal signs. No changes were observed for pain intensity (paired t-test). NRS=Numerical Rating Scale; ROE=Relative Opioid Equivalents; SOWS=subjective opioid withdrawal scale.

TABLE 16

| | | | |
|---|---|---|---|
| Baseline | Abstinence | | |
| (mean ± s.d.) | (mean ± s.d.) | T-value | p-value |

Regions that exhibited increased ALFF (CBP + O > CBP − O)

| | | | | |
|---|---|---|---|---|
| right pITG | 1.04 ± 0.02 | 1.06 ± 0.02 | −1.92 | 0.08 |
| left pITG | 1.07 ± 0.03 | 1.03 ± 0.03 | 3.39 | 0.004* |
| mPFC | 1.01 ± 0.02 | 0.96 ± 0.02 | 1.89 | 0.09 |
| right LOC | 1.03 ± 0.03 | 1.04 ± 0.03 | −1.28 | 0.15 |
| MCL | 1.04 ± 0.04 | 1.02 ± 0.03 | 1.22 | 0.24 |

Regions that exhibited decreased ALFF (CBP + O < CBP − O)

| | | | | |
|---|---|---|---|---|
| right aMTG | 0.99 ± 0.03 | 0.89 ± 0.04 | 6.78 | <10⁻⁴* |
| left aMTG | 0.96 ± 0.03 | 0.94 ± 0.02 | 1.84 | 0.09 |
| left dlPFC | 0.97 ± 0.02 | 0.98 ± 0.01 | −0.95 | 0.36 |

Table 16 shows regional ALFF changes, between before and following opioid abstinence, in 14 patients on long-term opioids. Most brain regions that showed significant ALFF differences between CBP+O and CBP−O showed minimal changes following opioid abstinence. Only the left pITG and right aMTG showed any significant change (paired t-test p<0.05). ALFF=Amplitude of low-frequency fluctuations; pITG=posterior inferior temporal gyrus; mPFC=middle prefrontal cortex; LOC=Lateral occipital cortex; MCL=mesocorticolimbic region; aMTG=anterior middle temporal gyrus; dlPFC=dorsolateral prefrontal cortex. *p<0.05 Bonferroni corrected.

Successful, and Unsuccessful tapering: the effect of opioid tapering in 21 CBP patients following a 4-week multi-disciplinary non-pharmacological chronic pain rehabilitation program was examined (Table 17). Out of the 21 patients, 8 patients decreased opioid use by more than 30% (tapered group), while 13 patients showed minimal/no change in medication use (non-tapered group, Table 18). Both groups' pain decreased following the treatment (FIG. 4A, last column, rows 3,4, Table 18), and most brain regions that showed significant ALFF differences between CBP+O and CBP−O showed minimal changes with treatment except for left pITG (Table 18). In the patients who tapered their opioid use, within-subject ΔALFF between before and after treatment tightly negatively correlated with 5-HT_{1A} and 5-HT_{1B} receptor distributions and positively correlated with MOR receptor distribution in the cortex (FIG. 4A, third row). In addition, post-treatment, increased 5-HT_{1A}R*ALFF and 5-HT_{1B}R*ALFF values was observed and decreased MOR*ALFF values, as compared to the pre-treatment values (FIG. 4D). In contrast, patients who did not taper their opioid use showed minimal to no changes in the three receptor-based parameters (FIGS. 4A and 4E).

TABLE 17

CBP
mean ± s.d.

Demographic and general health characteristics

| | |
|---|---|
| Age (years) | 49.33 ± 15.20 |
| Sex/female (%) | 16 (76.19%) |

Pain characteristics

| | |
|---|---|
| Pain intensity (NRS) | 6.71 ± 1.84 |
| Pain duration (years) | 13.51 ± 11.01 |

Opioid consumption and behavior

| | |
|---|---|
| MME | 13.81 ± 15.77 |

Table 17—Patient demographics and general health for patients who underwent 4-week multidisciplinary pain rehabilitation program. Patients had similar pain intensity and duration as well as age and sex distributions to CBP+O. Patients had lower MME values compared to CBP+O, with highest MME=51.7. MME=Morphine Milligram Equivalents.

TABLE 18

| | | | | | Group × |
|---|---|---|---|---|---|
| | | | | Treatment | Treatment |
| Tapered (n = 8) | | Non-tapered (n = 13) | | Effect | Effect |
| Pre | Post | Pre | Post | F-value | F-value |
| (mean ± s.d.) | (mean ± s.d.) | (mean ± s.d.) | (mean ± s.d.) | (P-value) | (P-value) |

Pain and opioid consumption

| | | | | | | | | |
|---|---|---|---|---|---|---|---|---|
| Pain intensity (NRS) | 7.25 ± 1.39 | 4.88 ± 1.64 | 6.38 ± 2.06 | 4.69 ± 2.62 | 14.33 | (0.001) | 0.40 | (0.53) |
| MME | 23.32 ± 18.10 | 13.11 ± 14.10 | 7.96 ± 11.23 | 8.01 ± 11.10 | 37.00 | (<10⁻⁶) | 37.6 | (<10⁻⁶) |

Regions that exhibited increased ALFF (CBP + O > CBP − O)

| | | | | | | | | |
|---|---|---|---|---|---|---|---|---|
| right pITG | 0.99 ± 0.02 | 0.99 ± 0.02 | 0.99 ± 0.02 | 0.99 ± 0.02 | 2.89 | (0.11) | 0.01 | (0.92) |
| left pITG | 1.03 ± 0.02 | 1.01 ± 0.03 | 1.01 ± 0.02 | 1.00 ± 0.02 | 8.80 | (0.007) | 0.03 | (0.87) |
| mPFC | 0.96 ± 0.01 | 0.96 ± 0.02 | 0.97 ± 0.03 | 0.98 ± 0.03 | 0.01 | (0.94) | 0.79 | (0.39) |
| right LOC | 0.98 ± 0.03 | 0.97 ± 0.03 | 1.01 ± 0.02 | 0.99 ± 0.02 | 2.73 | (0.11) | 0.34 | (0.56) |
| MCL | 0.98 ± 0.02 | 0.97 ± 0.02 | 0.96 ± 0.02 | 0.96 ± 0.02 | 0.35 | (0.56) | 0.00 | (0.94) |

Regions that exhibited decreased ALFF (CBP + O < CBP − O)

| | | | | | | | | |
|---|---|---|---|---|---|---|---|---|
| right aMTG | 1.07 ± 0.02 | 1.07 ± 0.02 | 1.07 ± 0.02 | 1.06 ± 0.02 | 1.89 | (0.18) | 0.09 | (0.77) |
| left aMTG | 1.05 ± 0.01 | 1.05 ± 0.01 | 1.05 ± 0.02 | 1.04 ± 0.01 | 3.90 | (0.06) | 0.02 | (0.88) |
| left dlPFC | 1.04 ± 0.02 | 1.04 ± 0.02 | 1.04 ± 0.02 | 1.04 ± 0.02 | 0.01 | (0.98) | 0.11 | (0.74) |

Table 18—Pain intensity, opioid consumption and regional ALFF changes following multidisciplinary pain rehabilitation program in patients who tapered or did not taper opioid use. Patients were divided into two groups based on changes in their opioid consumption (MME) following treatment (Group1=tapered, >30% MME decrease following treatment; Group2=non-tapered <30% MME decrease following treatment). Out of the 21 patients, 8 tapered their opioid use. Changes in pain, MME and regional ALFF changes were assessed using a repeated measure ANOVA. Pain intensity showed a significant treatment effect, but no group X treatment interaction, indicating that pain intensity decreased similarly for tapered and non-tapered patients. MME showed both a treatment and group x treatment effects. Most brain regions that showed significant ALFF differences between CBP+O and CBP−O showed minimal changes following treatment for tapered and non-tapered groups. Only the left pITG showed a significant treatment effect. No region showed any significant Group x treatment effect. ALFF=Amplitude of low-frequency fluctuations; pITG=posterior inferior temporal gyrus; mPFC=middle prefrontal cortex; LOC=Lateral occipital cortex; MCL=mesocorticolimbic region; aMTG=anterior middle temporal gyrus; dlPFC=dorsolateral prefrontal cortex; MME=Morphine Milligram Equivalents.

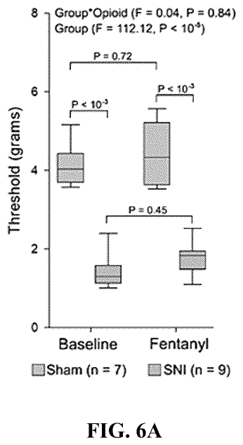

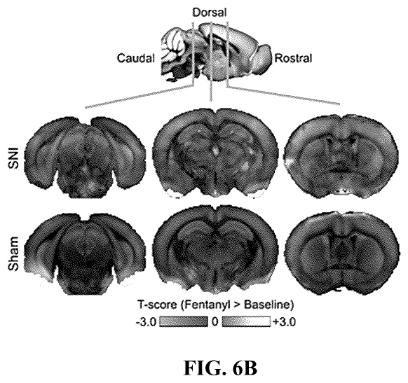

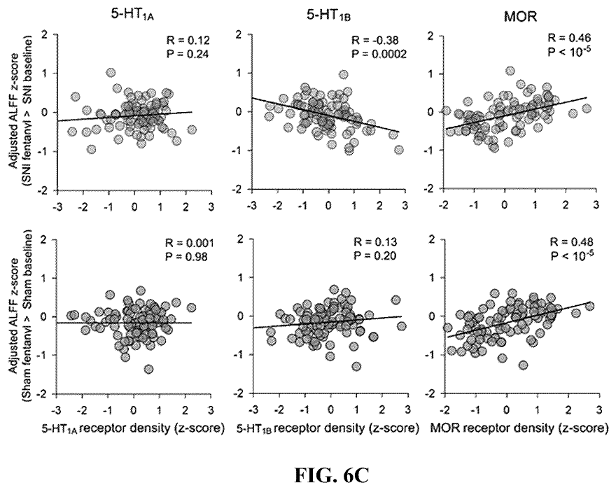

To establish across species and experimental generalizability of the disclosed results, it was tested whether or not the three receptor-based adaptations can be observed in a mouse model of long-term opioid exposure. Resting-state brain activity was collected in 9 chronic neuropathic pain model mice (spared nerve injury, SNI) and in 7 sham injured mice before and after 20 days of daily fentanyl vapor self-administration (fentanyl exposure started 1 month after the peripheral injury). Pain-like behavior was assayed by testing tactile withdrawal thresholds for the injured paw in SNI and Sham mice. Withdrawal thresholds for the injured paw were lower in SNI compared to sham and did not change following fentanyl (tested 24 hours after fentanyl vaping) (FIG. 6A).

Mouse brain expression profiles were obtained from the Allen Atlas [46], for 18 of the 19 receptors and transporters that were studied in humans. Mouse ΔALFF was calculated separately for Sham and SNI animals (FIG. 6B) and modeled the result in multiple regression using all 18 receptor expression maps for the entire brain (92 regions). Similar to our human results, in SNI animals (chronic pain model) ALFF changes following fentanyl exposure were negatively related to 5-HT_{1B} and positively related to MOR expressions in the brain. However, ΔALFF was not associated with 5-HT_{1A} in SNI mice (FIG. 6C), which may be at least partially attributable to the effects of anesthesia on serotonin-related activity [47]. In contrast to SNI, ΔALFF changes in sham animals were only related to MOR, but not to 5-HT_{1A} and 5-HT_{1B} (FIG. 6C).

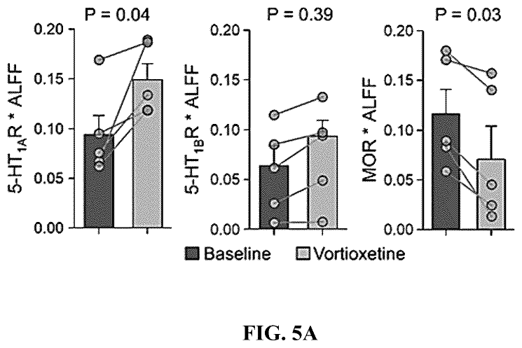

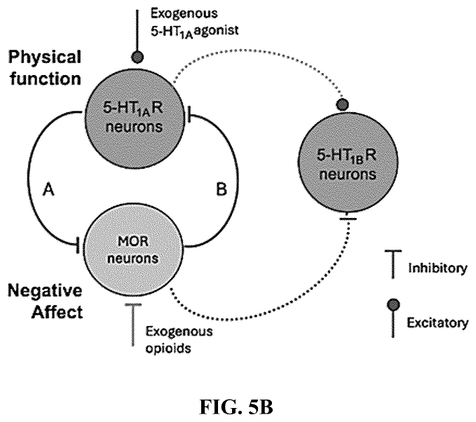

Perturbations show that serotonin receptor- and MOR-related activities are divergent. Such a result is simply that the 5-HT_{1A}R neuronal population may inhibit the MOR population. This concept was tested in 5 healthy volunteers by examining brain activity ΔALFF (post-pre) after ingesting a single dose of a 5-HT_{1A} agonist (vortioxetine). In all subjects, increased 5-HT_{1A}R*ALFF and decreased MOR*ALFF, and no change in 5-HT_{1B}R*ALFF (FIG. 5A) was observed. The specificity of the result is remarkable, as 15 of the remaining 16 receptor-related activities remained unchanged (only dopamine D1R*ALFF changed with vortioxetine ingestion). The data confirms the hypothesis and suggests that 5-HT_{1A}R neuronal populations compete with MOR neuronal populations. Thus, serotonergic agonists may counteract the effects of long-term opioid use and help in opioid tapering and even in improving chronic pain.

The results can be summarized in a circuit motif model of a receptor-specific brain-wide dynamical system of long-term opioid exposure (FIG. 5B). The primary concept is that long-term opioid use in chronic pain patients drives cortical (a) hyperactivity of opioidergic circuits and (b) hypoactivity of cortical serotonergic (5-HT_{1A}R and 5-HT_{1b}R) circuits. It is well-established that opioid use drives mu-opioid receptor (MOR) downregulation. Since MORs are inhibitory, downregulation hyperactivates MOR-containing neurons. It was observed that this hyperactivity is associated with blunted 5HTergic activity. The competitive interaction between MOR and 5-HT_{1A}R neuronal pools is rapid (within a day), as previously observed in abstinence and vortioxetine results, while longer-term adaptations (weeks) control the 5-HT_{1B}R neuronal pool interaction with MOR and 5-HT_{1A}R neurons, as seen for successful tapering.

The present disclosure uncovered how long-term opioid consumption in chronic back pain is associated with specific neurobiological outcomes. Given that chronic pain and OUD show similar psychological comorbidities and brain structural and functional maladaptations, the expectation was that CBP+O [chronic back pain (CBP) managed with opioids (+O)] would substantially deviate from CBP−O [chronic back pain (CBP) managed without opioids (−O)]—i.e., opioid-related adaptations would be additive to chronic pain-related adaptations. Behaviorally, long-term opioid use was associated with only modestly poorer outcomes than CBP−O. At the same time, evidence for any benefits of long-term opioid use was not found. Structurally, opioid use was associated with lower whole-brain gray matter volume and lower regional gray matter density than CBP−O. These structural changes were also modest compared to those observed in CBP [39,40] or substance use disorder (SUD) [41,42].

Systematic reviews show that long-term opioid use has minimal efficacy for chronic pain [48-51], and opioids are not superior to non-steroidal anti-inflammatories when used long-term in chronic pain management [52-55]. However, there are also critical dissenting views claiming that opioids may help manage chronic pain, that opioid use may be relatively safe, and that their abuse potential remains unclear even when used at very high doses [56]. Long-term opioid exposure in CP can result in physiological dependence, opioid-induced hyperalgesia, OUD, increased risk of fractures, delirium, dementia, and other complications [8, 38, 57, 58]. The results of the current study are clear. It was not demonstrated that long-term opioid consumption modulates chronic pain. Numerical ratings of back pain (NRS) matched between CBP−O and CBP+O were similarly related to PC2-Pain quality in both groups (Table 3), and PC2 was not modulated by opioid use (MME), blood levels (ROE), and duration of use(DOU) (FIG. 1F), discounting the influence of opioid use on PC2. The 5-HT_{1A} and MOR distributions in the cortex were related to PCi and PC3 but not to PC2 (FIGS. 3D-3E). More importantly, back pain intensity ratings between high and low opioid users, and before and after opioid abstinence, were not different, and back pain intensity similarly decreased in subjects who tapered or did not taper their opioids (FIG. 4A, last column). Complementarily, it was observed that neuropathic mice who consumed fentanyl for two weeks showed no long-term change in tactile sensitivity of the injured paw from before fentanyl use (analgesia was observed in the few hours post fentanyl use, data not shown). Thus, although chronic pain patients may experience pain relief when they start on opioids, their persistent consumption of opioids does not seem to be driven by the need to control their ongoing pain. Instead, the evidence shows that long-term opioid use further worsens the functional disability (further worsened by high levels of opioids in the blood, FIG. 1F, FIG. 1I, and with high BMI, Table 3) and negative affect that already exist in CBP−O (FIGS. 1A-1B and Table 2). But the influence of confounding by indication regarding the cross-sectional comparisons cannot be ruled out.

In contrast to the small behavioral and brain morphology changes, large differences in spontaneous brain activity was observed between CBP+O and CBP−O. The motivational, affective MCL circuit exhibited the most prominent increased ALFF activity in CBP+O. In contrast, the dlPFC, which is suggested to provide cognitive control over the MCL circuits [59], showed the largest decrease in activity (FIG. 2A, Table 11). According to the canonical view, the MCL system signals both the reward value and associated reward expectations. However, recent evidence [19,60] and human fMRI [61,62] and rodent studies [63,64] implicate this circuit in aversive states and chronic pain. Yet regional peak brain activity changes were not related to opioid use or to clinical parameters (Table 12). Instead, whole-cortex receptor expression-related activity, 5-HT₁ₐR*ALFF and MOR*ALFF, respectively, reflected functional disability and negative affect for all 140 CBP−O and CBP+O (FIGS. 3D-3E).

By modeling whole-cortex brain activity using 19 receptor expression distributions, a remarkably strong, almost uniform, relationship between three receptors and brain activity changes with long-term opioid exposure was uncovered (FIGS. 2D-2E), including their receptor-weighted activity (R*ALFF) and their interactions that distinguish between healthy subjects, CBP−O, and CBP+O (FIGS. 3A-3F). This novel multi-receptor approach of assaying brain activity may have utility in unraveling molecular mechanisms of diverse neurological conditions. In healthy subjects, 5-HT_{1A} and 5-HT_{1b} enhanced while MOR decreased ongoing activity, and this activity was not correlated across individuals. In chronic pain, MOR inhibition increased, and between-subject correlations were positively strengthened between 5-HT_{1A} and 5-HT_{1b} and negatively strengthened between MOR and both 5-HT_{1A} and 5-HT_{1b}. With long-term opioid use, the influence of 5-HT_{1A} and 5-HT_{1B} on brain activity diminished, but MOR's influence was enhanced (FIG. 2E, FIGS. 3A, 3C, and 3F). Changes in the activity and correlations between these three receptors for different subgroupings and different perturbations of opioids provide evidence for their causal role with opioid exposure (FIGS. 4A-4E), with approximate correspondences that was observed in a mouse model for chronic pain and long-term opioid exposure (FIGS. 6A-6C). The vortioxetine experiment is a proof-of-concept test (FIG. 5A). It demonstrates the sensitivity of the measures disclosed herein, the specificity of the molecular predictions derived from the results disclosed herein, the causal nature of the present observations, and, importantly, points to a clinically actionable molecular target and a potential drug to counteract the circuit adaptations of long-term opioid use that are associated with the functional disability and negative affect in both CBP−O and CBP+O.

The final model that summarizes the results disclosed herein (FIG. 5B) points to the dynamic interaction between three distinctly distributed neuronal pools. The weighted sum of which would approximate the brain activity change observed for opioid use (ΔALFF for CBP+O-CBP−O), where the MOR pool has the highest density in mPFC, part of the MCL, 5-HT_{1A}R pool has the highest density in mid temporal gyrus, and 5-HT_{1B}R pool has the highest density in anterior temporal regions and lowest density around DLPFC. The two competing systems are mutually inhibitory. In healthy subjects, activities in the three neuronal pools are not correlated across subjects. However, with long-term opioid use, MOR-s are desensitized, disinhibited by MCL activity, and inhibit the 5HT_{1A} and 5HT_{1B} systems by strengthening the B pathway. The latter is maintained by a regular and cyclic exogenous opioid supply, a state dominated by avoidance of negative affect (akin to controlling hyperkatifeia in stark OUD [22]) at the cost of increased functional disability. As CBP+O subjects are on a regular regimen of opioid use, the extent of underlying OUD remains hard to establish. Yet, the activity pattern can be interpreted as diminished cognitive control leading to oscillations between worsening negative affect or functional disability.

It has been repeatedly demonstrated herein that regional brain activity or structural properties are minimally informative regarding the clinical implications of opioid use. In contrast, whole-cortex activity patterns capture opioid use properties. Moreover, it was observed that vastly different activity pattern changes (e.g., FIG. 4A, first column) can be conceptualized as cortex-wide receptor-related activity changes. These observations reinforce the importance of brain-wide activity [30] by demonstrating its clinical role.

The methods disclosed herein have important clinical implications. Surprisingly, the inventors showed the existence of chronic pain patients who take prescription opioids for long periods (at least for the types of opioids included here, and irrespective of dosage) with minimal negative impact on their psychology and brain structure. Thus, there is no compelling reason why such patients should undergo opioid tapering, particularly since these patients often have trouble reducing their dose. Patients' trouble with tapering may come as a surprise given the lack of brain structural differences relative to CBP−O, perhaps suggesting it may be more attributable to the long-term effects of opioids on brain function and related molecular adaptations. The remarkable specificity between activity changes and receptor-type expression levels observed here suggests the utility of specific chemicals to control particular parts of the circuitry.

### Experiment Procedures

The first portion of this disclosure is a case-control assessment of the effect of opioid consumption on neuropsychology in subjects on steady and long-duration opioid consumption to manage their CBP(CBP+O) in contrast to CBP subjects not using opioids (CBP−O). Clinical phenotypic properties were collected and contrasted between the groups using 17 questionnaire outcomes. Opioid use was assessed based on drug prescription, blood concentration, and duration of opioid consumption. The risk was evaluated of opioid misuse and the intensity of opioid withdrawal signs. Dependence of clinical parameters on opioid use, opioid misuse, and withdrawal risk were also assessed. All participants underwent brain scans to collect anatomical and functional (resting state-fMRI) data. Brain grey matter and white matter were contrasted between the groups and related to opioid use, opioid misuse, and withdrawal risk. Spontaneous brain activity (based on Amplitude of low-frequency fluctuations, ALFF, was contrasted between the two groups. The resultant cortical map was modeled with 19 neurotransmitter receptor density maps (constructed from PET images of 1,238 healthy participants).

In the second part of the disclosure, the receptor-related results of the case-control assessment were assessed regarding their dependence on various manipulations. Subgroups of CBP+O with high and low opioid use were contrasted with each other. In a subsample of the CBP+O, brain activity was collected a second time after a brief period of stopping opioid consumption. A different group of CBP+O underwent a 4-week rehabilitation treatment, and their brain activity changes pre- and post-treatment were assessed as a function of opioid tapering, relative to receptor-related activities. To test the generalizability of results to mice, the same receptor-related brain activity changes were studied with exposure to fentanyl vapor self-administration in sham and chronic neuropathic model animals. Moreover, in healthy subjects, changes in receptor-related brain activity were tested between prior and after ingestion of a single dose of a 5-HT_{1A} agonist.

70 CBP patients on opioid therapy for at least 6 months (CBP+O) were recruited and 70 patients not taking opioids (CBP−O) matched for age, sex, pain intensity, and pain duration. Recruitment was from the Northwestern Medicine (NM) healthcare system and Shirley Ryan Ability Lab. The exclusion criteria included (1) treatment with a spinal cord stimulator; (2) diagnosis of rheumatoid arthritis, ankylosing spondylitis, acute vertebral fractures, fibromyalgia, low back/spine oncologic history, and other comorbid neurological disorders, including major depression and psychiatric disorder requiring treatment; (3) involvement in litigation regarding their back pain, having a disability claim, or receiving workman's compensation; (4) significant comorbid diseases such as uncontrolled hypertension, unstable diabetes mellitus, renal insufficiency, congestive heart failure, coronary or peripheral vascular disease, chronic obstructive lung disease, or malignancy; (5) Pregnancy during the study. Twenty-one CBP+O subjects were recruited from Shirley-Ryan Ability Lab to study the effects of a non-pharmacological treatment on receptor-related brain activity changes with or without opioid tapering. Five healthy subjects were recruited from the community to examine the effects of ingesting Vortioxetine on receptor-related brain activity changes. All participants completed a personal health history questionnaire, including information about age, gender, race, ethnicity, height, weight, alcohol use, and smoking status.

Pain duration and pain characteristics were recorded for all participants. Pain characteristics were assessed using the Numerical Rating Scale NRS, McGill Pain Questionnaire-Short Form (sf-MPQ), and Pain Detect. The NRS is an 11-point numerical rating scale used to measure pain intensity, with “0” corresponding to no pain and “10” to the worst pain possible (or imaginable) 65. The sf-MPQ is 15 item measure that separates the sensory and affective components of pain 66. Finally, the PainDETECT consists of seven items assessing for neuropathic pain 67. Also, the Pain Catastrophizing Scale (PCS) was collected, a survey with 13 items asking about thoughts or feelings related to pain experiences [68].

Mood and emotional measures included the Beck Depression Inventory (BDI), a 21 multiple choice self-reported questionnaire evaluating the severity of depression [69], the Positive and Negative Affect (PANAS): 20 items self-report questionnaire measuring positive and negative affect [70], and the Pain Anxiety Symptoms Scale (PASS): 20 questions measuring chronic pain related anxiety and fear [71].

The Patient-Reported Outcomes Measurement Information System (PROMIS) was also collected, A 57 items questionnaire evaluating important health-related quality of life domains assessing the seven domains of: pain interference, fatigue, depression and sadness, anxiety and fear, sleep disturbance, physical function, and social activity [72,] as well as the Short Form Health Survey (SF-12), a self-reported questionnaire to evaluate the impact of health on one's quality of life. It results in two physical and mental health scores [73]. Finally, disability was assessed using Oswestry Low Back Pain Disability (ODI), a ten-item questionnaire measuring functional disability in activities of daily living in patients with low backpain [74].

The internal structure and multidimensionality of pain and behavioral measures were assessed using principal component analysis (PCA) in Matlab (R2020a, Statistics and Machine Learning Toolbox Version 11.7) with varimax rotations across all patients. Principal components with eigenvalue >1 and that explained >10% of variance were retained. To ensure that the components were not related to opioid use, PCA for CBP+O and CBP−O were performed separately. Similarity between the principal components determined from different groups were assessed by comparing their corresponding loading factors using correlation analysis.

For the group using opioids, details of their opioid prescription was collected. The daily dosage to morphine milligram equivalent (MME) was converted using the standard conversion factors generated by the CDC [75]. In addition, the duration of opioid use (DOU), and blood samples were collected immediately afterbrain scans. The concentrations of eight analytes (oxymorphone, hydromorphone, oxycodone, hydrocodone, fentanyl, buprenorphine, methadone, and tramadol), and the presence of three others (morphine, codeine, and heroin) in plasma were determined by liquid chromatography-tandem mass spectrometry. Samples were run in duplicate, and the average opioid concentration was used. The various opioid concentrations mg/L were then converted to a relative opiate equivalent (ROE), taking into consideration the affinity of each opioid to the mu-opioid receptor (MOR) [76] and the respective molar weights. Samples with peak presence of opioids below the lower limit of quantitation were imputed as the halfway point from zero to the measurable lower quantifiable threshold.

The risk of opioid misuse measured with the Current Opioid Misuse Measure (COMM) and the intensity of opioid withdrawal using the Subjective Opioid Withdrawal Scale (SOWS) was also evaluated

Participants were scanned with a 3 Tesla Siemens Magnetom Prisma whole-body scanner using a 64channel-head/neck coil. TI-anatomical brain images were acquired using a voxel size of 1×1×1 mm3, a repetition time/echo time (TR/TE)=2.3 s/2.4 ms, a flip angle of 9°, the in-plane resolution=256×256. Resting-state fMRI images were acquired using a voxel size of 2×2×2 mm3, a TR/TE=555 ms/22 ms, a flip angle of 47°. Diffusion tensor images were acquired with a two-shell dMRI protocol: the first acquisition had 64 directions and a bval=2000, and the second with 30 directions and a bval=700. Both had a voxel size of 2×2×2 mm3, a TR/TE=3.5 s/9.2 ms, and a flip angle 90.

Preprocessing of resting-state fMRI images was performed using the standardized FMRIPREP pipeline. Following the removal of the first 100 volumes to eliminate saturation effects and achieve steady-state magnetization, the following steps were performed: motion correction, intensity normalization, nuisance regression of 6 motion vectors, signal-averaged overall voxels of the eroded white matter and ventricle region, and global signal of the whole brain. All pre-processed fMRI data was registered to the MNI1522 mm template using non-linear registration in FSL.

ALFF analysis 45 was performed on preprocessed data using Matlab software. The time series for each voxel was transformed to the frequency domain, and the power spectrum was then obtained across 0.08-0.1 Hz at each voxel, divided by the global mean of ALFF within a brain mask. Spatial smoothing was then applied using an isotropic Gaussian kernel of 5 mm full width at half-maximum. Differences in ALFF between groups were performed using randomize in FSL. Correction for multiple comparisons was performed using the Threshold-Free Cluster Enhancement (TFCE) method (FWEp<0.01).

Regions of interest (ROI) that showed significant ALFF changes between groups were determined post-hoc from the whole-brain ALFF contrast map using Easythresh.

Peripheral gray matter volume (PGMV) for each subject was estimated using SIENAX in FSL. It is an automated process that utilizes FSL programs to strip the non-brain tissue from the estimated peripheral gray matter volume (PGMV).

Gray matter density (GMD) was examined using voxel-based morphometry from FSL-VBM. All T1-weighted images were first brain extracted and then segmented into gray matter, white matter, or cerebrospinal fluid. A common gray matter template was generated for patients by registering and averaging all gray matter images. The gray matter image of each participant was then registered to the common template using non-linear transformation. Differences between groups (CBP+O>CBP−O) was performed using randomize in FSL, with age, MQS, sex, race, pain intensity, pain duration (log) and intercranial volume as regressors. Correction for multiple comparisons were performed using Threshold-Free Cluster Enhancement (TFCE) method (FEW p<0.01).

Volumes of subcortical regions were obtained using FSL. The T1 images were segmented using FSL-FIRST with no boundary correction. Volumes of subcortical regions were determined using a voxel count. Differences in subcortical volumes between CBP+O and CBP−O was determined using ANCOVA analysis with age, sex, race, pain intensity, pain duration (log), BMI, MQS and intracranial brain volume as regressors.

Fractional anisotropy (FA) maps were used as a proxy for white-matter integrity. DTI analyses were conducted using tools from the FMRIB diffusion toolbox (FDT). First, diffusion-weighted images were visually inspected for gross artifacts. Images were then corrected for eddy current distortions and head movement using EDDY [77]. For additional unbiased quality control, QUAD and SQUAD [78] was used. DTIFIT was used to fit a tensor to the data and calculate principal directions, and to further extract fractional anisotropy maps. To perform group-level analyses, FA images were analyzed with Tract-Based Spatial Statistics [79](TBSS), which provides good inter-subject alignment for white-mater images. Diffusion images were transformed into standard space (FMRIB58) through non-linear transformation, and then projected to the mean skeletonized images, thresholded at the default value of FA=0.2. Statistical analyses were conducted using the same statistical design and methods as for VBM analyses plus an additional motion confound as a covariate of no interest (relative motion estimated by QUAD). Statistical analyses were also conducted with randomize with TFCE cluster enhancement (optimized for TBSS analyses, option-T2) and FWE correction for multiple comparisons.

The unthresholded whole-brain contrast map ALFF was parcellated to 100 cortical brain regions according to the Schaefer atlas [80]. The relationship of the regional change in ALFF z-scores with the corresponding z-scores of 19 different neurotransmitter receptors and transporters density maps obtained from Hansen et al. [31] was determined using a multiple regression analysis.

In the experimental design for multi-disciplinary non-pharmacological rehabilitation treatment, a total of 25 patients were enrolled in the study. Four patients did not complete the study: two withdrew from the study and two were discharged early due to non-adherence and medical complications. The study completers did not differ from the total sample of patients who enrolled based on mean age (t=−0.230, p=0.819) or pain duration (t=−0.322, p=0.749) or distributions of sex (χ²=0.000, p=0.988) and primary diagnoses (χ²=1.281, p=0.973).

Patients participated in a full day (8 hours per day, 5 days per week) program for four weeks. The program included both individual and group therapy sessions. Individual sessions included pain psychology, physical therapy, occupational therapy, biofeedback/relaxation training, and medical management. All brain imaging parameters and analysis were similar to methods used for the case-control study.

Animal experiments were conducted using adult male and female C57BL/6 mice aged 10-12 weeks attained from Jackson Labs. Mice were maintained on a 12/12-hour light/dark cycle. Both food and water were provided ad libitum. Mice were given either spared nerve injury (SNI) or sham. Mice were anesthetized with isoflurane (2-3%) during the procedure. For SNI, an incision was made on the left leg, and the sciatic nerve was exposed. Both the common peroneal and tibial nerve was ligated and tied with 6-0 vicryl sutures, while the sural nerve remained intact. For the sham group, the nerve was left intact.

Experiments were conducted 1 month after surgery. Mice were scanned at two time points: prior to fentanyl exposure and two weeks after fentanyl consumption. Fentanyl self-administration sessions occurred 5 days a week, separated by 2 days. The mice were trained to self-administer fentanyl on a fixed-ratio (FR) schedule, FR1 for the first 5 days and FR5 for the next 5 days, followed by a second brain scan, which was collected 1 day after the last self-administration session. Mechanical allodynia data was also collected just before the brain scans.

Fentanyl vapor self-administration sessions lasted 2 hours. Mice were placed into an enclosed Plexiglass chamber (14 cm×20 cm×23 cm) located within a sound-attenuating cabinet (MedAssociates). There were two nosepoke entry ports on either side of the chamber. During each session, nosepokes to the active port would result in a fentanyl vapor delivery for 2 seconds, while nosepokes to the inactive port would yield no consequences. Following a fentanyl vapor delivery, there was a 1-minute timeout period in which active nosepokes were inconsequential.

For collection of mechanical allodynia, mice were placed in a Plexiglass box with a grid floor. A MouseMet eVF electronic von Frey (Topcat Metrology Ltd, UK) was used to record the force of paw withdrawal. Each measure was recorded at least 3 times, per hind limb paw, and then averaged across trials.

5 mg/mL fentanyl hydrochloride (5 mg/ml) was dissolved in 20% propylene glycol and 80% glycerol. Fentanyl was obtained from the National Institute on Drug Abuse (NIDA Intramural Research Program Pharmacy (Baltimore, MD, USA).

MRI acquisition was conducted on a Bruker Clinscan 7T MRI. Mice were anesthetized with isoflurane and sedated with 0.3 mg/kg medetomidine. During each scan, both a resting state and a fentanyl task scan were collected. Each scan lasted 10 minutes and consisted of 240 volumes (TR=2500 ms). Only resting state data was analyzed for this study.

In-situ hybridization data for selected genes were downloaded from the Allen Brain Institute using Python scripts to access their API. For this analysis, the data that was used was receptor “energy”, defined as the highest probability of gene expression calculated as: within a given area A (voxel or structure), expression energy=(sum of intensity of expressing pixels in A)/(sum of all pixels in A). Data was downloaded for selected receptors available as coronal slices. Data was extracted in MHiD format and converted to NIFTI format and transformed them to standard space for analyses.

The present disclosure is described above and in the accompanying drawings with reference to a variety of examples. The purpose served by the disclosure, however, is to provide examples of the various features and concepts related to the disclosure, not to limit the scope of the disclosure. One skilled in the relevant art will recognize that numerous variations and modifications may be made to the examples described above without departing from the scope of the present disclosure.

### REFERENCES

[1] Ashaye, T. et al. Opioid prescribing for chronic musculoskeletal pain in UK primary care: results from a cohort analysis of the COPERS trial. BMJ Open 8, e019491 (2018). https://doi.org:10.1136/bmjopen-2017-019491
[2] Voon, P., Karamouzian, M. & Kerr, T. Chronic pain and opioid misuse: a review of reviews. SubstAbuse Treat Prev Policy 12, 36 (2017). https://doi.org:10.1186/s13011-017-0120-7
[3] Dahlhamer, J. M., Connor, E. M., Bose, J., Lucas, J. L. & Zelaya, C. E. Prescription Opioid Use AmongAdults With Chronic Pain: United States, 2019. Natl Health Stat Report, 1-9 (2021).
[4] Hoy, D. et al. A systematic review of the global prevalence of low back pain. Arthritis Rheum 64, 2028-2037 (2012). https://doi.org:10.1002/art.34347
[5] Wu, A. et al. Global low back pain prevalence and years lived with disability from 1990 to 2017: estimates from the Global Burden of Disease Study 2017. Ann Transl Med 8, 299 (2020). https://doi.org:10.21037/atm.2020.02.1756.
[6] Caldeira-Kulbakas, M., Stratton, C., Roy, R., Bordman, W. & Mc Donnell, C. A prospective observational study of pediatric opioid prescribing at postoperative discharge: how much is actually used? Can J Anaesth 67, 866-876 (2020). https://doi.org:10.1007/s12630-020-01616-5
[7] Kelley-Quon, L. I. et al. Guidelines for Opioid Prescribing in Children and Adolescents After Surgery: An Expert Panel Opinion. JAMA Surg 156, 76-90 (2021). https://doi.org:10.1001/jamasurg.2020.50458.
[8] Volkow, N. D. & McLellan, A. T. Opioid Abuse in Chronic Pain—Misconceptions and Mitigation Strategies. N Engl J Med 374, 1253-1263 (2016). https://doi.org:10.1056/NEJMra1507771
[9] Medicine, I. o. Relieving pain in America: a blueprint for transforming prevention, care, education, and research. <http://www.iom.edu> (2011).
[10] Florence, C., Luo, F. & Rice, K. The economic burden of opioid use disorder and fatal opioid overdosein the United States, 2017. Drug Alcohol Depend 218, 108350 (2021). https://doi.org:10.1016/j.drugalcdep.2020.108350
[11] Baliki, M. N. & Apkarian, A. V. Nociception, Pain, Negative Moods, and Behavior Selection. Neuron 87,474-491 (2015). https://doi.org:10.1016/j.neuron.2015.06.005
[12] Herlinger, K. & Lingford-Hughes, A. Opioid use disorder and the brain: a clinical perspective. Addiction 117, 495-505 (2022). https://doi.org:10.1111/add.15636
[13] Tolomeo, S., Steele, J. D., Ekhtiari, H. & Baldacchino, A. Chronic heroin use disorder and the brain: Current evidence and future implications. Prog Neuropsychopharmacol Biol Psychiatry 111, 110148(2021). https://doi.org:10.1016/j.pnpbp.2020.110148
[14] Stewart, J. L., May, A. C. & Paulus, M. P. Bouncing back: Brain rehabilitation amid opioid and stimulant epidemics. Neuroimage Clin 24, 102068 (2019). https://doi.org:10.1016/j.nicl.2019.102068
[15] Ieong, H. F. & Yuan, Z. Resting-State Neuroimaging and Neuropsychological Findings in Opioid Use Disorder during Abstinence: A Review. Front Hum Neurosci 11, 169 (2017). https://doi.org:10.3389/fnhum.2017.00169
[16] Wollman, S. C. et al. Gray matter abnormalities in opioid-dependent patients: A neuroimaging meta-analysis. Am J Drug Alcohol Abuse 43, 505-517 (2017). https://doi.org:10.1080/00952990.2016.1245312
[17] Koob, G. F. & Volkow, N. D. Neurobiology of addiction: a neurocircuitry analysis. Lancet Psychiatry 3, 760-773 (2016). https://doi.org:10.1016/S2215-0366(16)00104-8
[18] Valentino, R. J., Nair, S. G. & Volkow, N. D. Neuroscience in addiction research. J Neural Transm (Vienna) (2023). https://doi.org:10.1007/s00702-023-02713-7
[19] de Jong, J. W., Fraser, K. M. & Lammel, S. Mesoaccumbal Dopamine Heterogeneity: What Do Dopamine Firing and Release Have to Do with It? Annu Rev Neurosci 45, 109-129 (2022). https://doi.org:10.1146/annurev-neuro-110920-011929
[20] Volman, S. F. et al. New insights into the specificity and plasticity of reward and aversion encoding in the mesolimbic system. J Neurosci 33, 17569-17576 (2013). https://doi.org:10.1523/JNEUROSCI.3250-13.2013 33/45/17569 [pii]
[21] Navratilova, E. & Porreca, F. Reward and motivation in pain and pain relief Nat Neurosci 17, 1304-1312 (2014). https://doi.org:10.1038/nn.3811
[22] Koob, G. F. Neurobiology of Opioid Addiction: Opponent Process, Hyperkatifeia, and Negative Reinforcement. Biol Psychiatry 87, 44-53 (2020). https://doi.org:10.1016/j.biopsych.2019.05.023
[23] Wakaizumi, K. et al. Psychosocial, Functional, and Emotional Correlates of Long-Term Opioid Use in Patients with Chronic Back Pain: A Cross-Sectional Case-Control Study. Pain Ther 10, 691-709(2021). https://doi.org:10.1007/s40122-021-00257-w
[24] Zortea, M. et al. Spectral Power Density analysis of the resting-state as a marker of the central effects of opioid use in fibromyalgia. Sci Rep 11, 22716 (2021). https://doi.org:10.1038/s41598-02101982-0
[25] Younger, J. W. et al. Prescription opioid analgesics rapidly change the human brain. Pain 152, 1803-1810 (2011). https://doi.org:10.1016/j.pain.2011.03.028
[26] Upadhyay, J. et al. Alterations in brain structure and functional connectivity in prescription opioid-dependent patients. Brain 133, 2098-2114 (2010). https://doi.org:10.1093/brain/awq138
[27] Murray, K., Lin, Y., Makary, M. M., Whang, P. G. & Geha, P. Brain Structure and Function of Chronic Low Back Pain Patients on Long-Term Opioid Analgesic Treatment: A Preliminary Study. Mol Pain 17, 1744806921990938 (2021). https://doi.org:10.1177/1744806921990938
[28] McConnell, P. A. et al. Impaired frontostriatal functional connectivity among chronic opioid using pain patients is associated with dysregulated affect. Addict Biol 25, e12743 (2020). https://doi.org:10.1111/adb.12743
[29] Kovoor, A., Nappey, V., Kieffer, B. L. & Chavkin, C. Mu and delta opioid receptors are differentially desensitized by the coexpression of beta-adrenergic receptor kinase 2 and beta-arrestin 2 in xenopus oocytes. J Biol Chem 272, 27605-27611 (1997). https://doi.org:10.1074/jbc.272.44.27605
[30] Vigotsky, A. D. J., R.; Branco, P.; Iannetti, G. D.; Baliki, M. N.; Apkarian, A. V. in Biorxiv (Biorxiv, Biorxiv, 2022).
[31] Hansen, J. Y. et al. Mapping neurotransmitter systems to the structural and functional organization of the human neocortex. Nat Neurosci 25, 1569-1581 (2022). https://doi.org:10.1038/s41593-022-01186-3
[32] Welsch, L., Bailly, J., Darcq, E. & Kieffer, B. L. The Negative Affect of Protracted Opioid Abstinence: Progress and Perspectives From Rodent Models. Biol Psychiatry 87, 54-63 (2020). https://doi.org:10.1016/j.biopsych.2019.07.027
[33] Chou, R. et al. The effectiveness and risks of long-term opioid therapy for chronic pain: a systematic review for a National Institutes of Health Pathways to Prevention Workshop. Ann Intern Med 162, 276-286 (2015). https://doi.org:10.7326/M14-2559
[34] Rached, G. V., A. D.; Branco, P.; Jabakhanji, R.; Schnitzer, T. J.; Apkarian, A. V.; Baliki, M. N. (Medrxiv, 2022).
[35] Wasan, A. D., Davar, G. & Jamison, R. The association between negative affect and opioid analgesia in patients with discogenic low back pain. Pain 117, 450-461 (2005). https://doi.org:10.1016/j.pain.2005.08.006
[36] Wasan, A. D. et al. Psychiatric Comorbidity Is Associated Prospectively with Diminished Opioid Analgesia and Increased Opioid Misuse in Patients with Chronic Low Back Pain. Anesthesiology 123, 861-872 (2015). https://doi.org:10.1097/ALN.0000000000000768
[37] Manchikanti, L. et al. Comprehensive, Evidence-Based, Consensus Guidelines for Prescription of Opioids for Chronic Non-Cancer Pain from the American Society of Interventional Pain Physicians (ASIPP). Pain Physician 26, S7-S126 (2023).
[38] Dowell, D., Haegerich, T. M. & Chou, R. CDC Guideline for Prescribing Opioids for Chronic Pain—United States, 2016. JAMA 315, 1624-1645 (2016). https://doi.org:10.1001/jama.2016.1464
[39] Tatu, K. et al. How do morphological alterations caused by chronic pain distribute across the brain?A meta-analytic co-alteration study. Neuroimage Clin 18, 15-30 (2018). https://doi.org:10.1016/j.nicl.2017.12.029
[40] Yuan, C. et al. Gray Matter Abnormalities Associated With Chronic Back Pain: A Meta-Analysis of Voxel-based Morphometric Studies. Clin J Pain 33, 983-990 (2017). https://doi.org:10.1097/AJP.0000000000000489
[41] Pando-Naude, V. et al. Gray and white matter morphology in substance use disorders: a neuroimaging systematic review and meta-analysis. Transl Psychiatry 11, 29 (2021). https://doi.org:10.1038/s41398-020-01128-2
[42] Yan, H. et al. Functional and structural brain abnormalities in substance use disorder: A multimodalmeta-analysis of neuroimaging studies. Acta Psychiatr Scand 147, 345-359 (2023). https://doi.org:10.1111/acps.13539
[43] Ding, J. R. et al. Topological fractionation of resting-state networks. PLoS One 6, e26596 (2011).
[44] Tomasi, D. & Volkow, N. D. Association between functional connectivity hubs and brain networks. Cereb Cortex 21, 2003-2013 (2011).
[45] Yang, H. et al. Amplitude of low frequency fluctuation within visual areas revealed by resting-state functional MRI. Neuroimage 36, 144-152 (2007). https://doi.org:10.1016/j.neuroimage.2007.01.054
[46] Sunkin, S. M. et al. Allen Brain Atlas: an integrated spatio-temporal portal for exploring the central nervous system. Nucleic Acids Res 41, D996-D1008 (2013). https://doi.org:10.1093/nar/gks1042
[47] Hamada, H. T. et al. Optogenetic activation of dorsal raphe serotonin neurons induces brain-wide activation. Nat Commun 15, 4152 (2024). https://doi.org:10.1038/s41467-024-48489-6
[48] Nury, E. et al. Efficacy and safety of strong opioids for chronic noncancer pain and chronic low backpain: a systematic review and meta-analyses. Pain 163, 610-636 (2022). https://doi.org:10.1097/j.pain.0000000000002423
[49] Trescot, A. M., Datta, S., Lee, M. & Hansen, H. Opioid pharmacology. Pain Physician 11, 5133-153(2008).
[50] Chou, R., Ballantyne, J. C., Fanciullo, G. J., Fine, P. G. & Miaskowski, C. Research gaps on use of opioids for chronic noncancer pain: findings from a review of the evidence for an American Pain Society and American Academy of Pain Medicine clinical practice guideline. J Pain 10, 147-159(2009). https://doi.org:10.1016/j.jpain.2008.10.007
[51] Chou, R. et al. Clinical guidelines for the use of chronic opioid therapy in chronic noncancer pain. J Pain 10, 113-130 (2009). https://doi.org:10.1016/j.jpain.2008.10.008
[52] White, A. P., Arnold, P. M., Norvell, D. C., Ecker, E. & Fehlings, M. G. Pharmacologic management of chronic low back pain: synthesis of the evidence. Spine (Phila Pa 1976) 36, S131-143 (2011). https://doi.org:10.1097/BRS.0b013e31822f178f
[53] Romano, C. L., Romano, D. & Lacerenza, M. Antineuropathic and antinociceptive drugs combination in patients with chronic low back pain: a systematic review. Pain Res Treat 2012, (2012). https://doi.org:10.1155/2012/154781
[54] Tauben, D. Nonopioid medications for pain. Phys Med Rehabil Clin N Am 26, 219-248 (2015). https://doi.org:10.1016/j.pmr.2015.01.005
[55] Chou, R. & Huffman, L. H. Medications for acute and chronic low back pain: a review of the evidence for an American Pain Society/American College of Physicians clinical practice guideline. Ann InternMed 147, 505-514 (2007).
[56] Nadeau, S. E., Wu, J. K. & Lawhern, R. A. Opioids and Chronic Pain: An Analytic Review of the Clinical Evidence. Front Pain Res (Lausanne) 2, 721357 (2021). https://doi.org:10.3389/fpain.2021.721357
[57] Arout, C. A., Edens, E., Petrakis, I. L. & Sofuoglu, M. Targeting Opioid-Induced Hyperalgesia in Clinical Treatment: Neurobiological Considerations. CNS Drugs 29, 465-486 (2015). https://doi.org:10.1007/s40263-015-0255-x
[58] Naples, J. G., Gellad, W. F. & Hanlon, J. T. The Role of Opioid Analgesics in Geriatric Pain Management. Clin Geriatr Med 32, 725-735 (2016). https://doi.org:10.1016/j.cger.2016.06.006
[59] Goldstein, R. Z. & Volkow, N. D. Dysfunction of the prefrontal cortex in addiction: neuroimaging findings and clinical implications. Nat Rev Neurosci 12, 652-669 (2011). https://doi.org:10.1038/nrn3119
[60] Sands, L. P. et al. Subsecond fluctuations in extracellular dopamine encode reward and punishment prediction errors in humans. Sci Adv 9, eadi4927 (2023). https://doi.org:10.1126/sciadv.adi4927
[61] Baliki, M. N., Geha, P. Y., Fields, H. L. & Apkarian, A. V. Predicting value of pain and analgesia: nucleus accumbens response to noxious stimuli changes in the presence of chronic pain. Neuron 66, 149-160 (2010). https://doi.org:S0896-6273(10)00171-6 [pii]10.1016/j.neuron.2010.03.002
[62] Baliki, M. N. et al. Parceling Human Accumbens into Putative Core and Shell Dissociates Encoding of Values for Reward and Pain. J Neurosci 33, 16383-16393 (2013). https://doi.org:10.1523/JNEUROSCI.1731-13.2013
[63] Ren, W. et al. The indirect pathway of the nucleus accumbens shell amplifies neuropathic pain. Nat Neurosci 19, 220-222 (2016). https://doi.org:10.1038/nn.4199
[64] Ren, W. et al. Adaptive alterations in the mesoaccumbal network after peripheral nerve injury. Pain162, 895-906 (2021). https://doi.org:10.1097/j.pain.0000000000002092
[65] Farrar, J. T., Young, J. P., Jr., LaMoreaux, L., Werth, J. L. & Poole, M. R. Clinical importance of changes in chronic pain intensity measured on an 11-point numerical pain rating scale. Pain 94, 149-158(2001). https://doi.org:10.1016/s0304-3959(01)00349-9
[66] Melzack, R. The short-form McGill pain questionnaire. PAIN 30, 191-197 (1987). https://doi.org:10.1016/0304-3959(87)91074-8
[67] Freynhagen, R., Baron, R., Gockel, U. & Tolle, T. R. painDETECT: a new screening questionnaire to identify neuropathic components in patients with back pain. Curr Med Res Opin 22, 1911-1920(2006). https://doi.org:10.1185/030079906X132488
[68] Sullivan, M. J. L., Bishop, S. & Pivik, J. The Pain Catastrophizing Scale: Development and Validation. Psychological Assessment 7, 524-532 (1996). https://doi.org:10.1037//I040-3590.7.4.524
[69] Beck, A. T., Steer, R. A., Ball, R. & Ranieri, W. F. Comparison of Beck Depression Inventories-IA and -IIin Psychiatric Outpatients. Journal of Personality Assessment 67, 588-597 (1996). https://doi.org:10.1207/s15327752jpa6703_13
[70] Watson, D., Clark, L. A. & Tellegen, A. Development and validation of brief measures of positive and negative affect: the PANAS scales. J Pers Soc Psychol 54, 1063-1070 (1988). https://doi.org:10.1037//0022-3514.54.6.1063
[71] McCracken, L. M., Zayfert, C. & Gross, R. T. The Pain Anxiety Symptoms Scale: development and validation of a scale to measure fear of pain. Pain 50, 67-73 (1992). https://doi.org:10.1016/0304-3959(92)90113-p
[72] Cella, D. et al. PROMIS® Adult Health Profiles: Efficient Short-Form Measures of Seven Health Domains. Value in Health 22, 537-544 (2019). https://doi.org:https://doi.org/10.1016/j.jval.2019.02.004
[73] Ware, J. E., Kosinski, M. & Keller, S. D. A 12-Item Short-Form Health Survey: Construction of Scales and Preliminary Tests of Reliability and Validity. Medical Care 34 (1996).
[74] Fairbank, J. C., Couper, J., Davies, J. B. & O'Brien, J. P. The Oswestry low back pain disability questionnaire. Physiotherapy 66, 271-273 (1980).
[75] Prevention, C. f D. C. a. Analyzing prescription data and morphine milligram equivalents (MME). (2021).
[76] Volpe, D. A. et al. Uniform assessment and ranking of opioid Mu receptor binding constants for selected opioid drugs. Regulatory Toxicology and Pharmacology 59, 385-390 (2011). https://doi.org:https://doi.org/10.1016/j.yrtph.2010.12.007
[77] Andersson, J. L. R. & Sotiropoulos, S. N. An integrated approach to correction for off-resonance effects and subject movement in diffusion MR imaging. NeuroImage 125, 1063-1078 (2016). https://doi.org:10.1016/j.neuroimage.2015.10.019
[78] Bastiani, M. et al. Automated quality control for within and between studies diffusion MRI data using a non-parametric framework for movement and distortion correction. NeuroImage 184, 801-812(2019). https://doi.org:10.1016/j.neuroimage.2018.09.073
[79] Smith, S. M. et al. Tract-based spatial statistics: voxelwise analysis of multi-subject diffusion data. Neuroimage 31, 1487-1505 (2006). https://doi.org:10.1016/j.neuroimage.2006.02.024
[80] Schaefer, A. et al. Local-Global Parcellation of the Human Cerebral Cortex from Intrinsic Functional Connectivity MRI. Cereb Cortex 28, 3095-3114 (2018). https://doi.org:10.1093/cercor/bhx179
[81] Tetreault P, Baliki M N, Baria A T, Bauer W R, Schnitzer T J, Apkarian A V. Inferring distinct mechanisms in the absence of subjective differences: Placebo and centrally acting analgesic underlie unique brain adaptations. Hum Brain Mapp. 2018; 39(5):2210-23. Epub 20180207. doi: 10.1002/hbm.23999. PubMed PMID: 29417694; PMCID: PMC5895516.
[82] Multi-receptor whole-cortex adaptations with long-term opioid use in chronic pain [Internet]2024.

What is claimed is:

1. A method for facilitating opioid tapering in a patient undergoing long-term opioid therapy, the method comprising:
administering a therapeutically effective amount of a pharmaceutical composition targeting serotonergic and opioidergic receptor activity;
modulating serotonin (5-HT1A, 5-HT1B) and mu-opioid receptor (MOR) activity to counteract opioid-induced neuroadaptive changes;
monitoring a patient's neurobiological response to the pharmaceutical composition; and
adjusting the administering of the pharmaceutical composition based on a response of the patient to facilitate opioid dose reduction while minimizing withdrawal symptoms and pain exacerbation.

2. The method of claim 1, wherein the pharmaceutical composition comprises a selective 5-HT1A agonist, a 5-HT1B modulator, or combinations thereof.

3. The method of claim 1, wherein the pharmaceutical composition further comprises a mu-opioid receptor partial agonist or antagonist to modulate opioid receptor desensitization and withdrawal effects.

4. The method of claim 1, wherein the pharmaceutical composition is administered in a controlled tapering protocol over a predetermined period.

5. The method of claim 1, further comprising assessing cortical receptor-related activity before and during the opioid tapering process using functional neuroimaging.

6. A pharmaceutical composition for aiding opioid tapering consisting of:
a therapeutically effective amount of a 5-HT1A agonist;
a therapeutically effective amount of a 5-HT1B modulator;
a mu-opioid receptor partial agonist or antagonist; and
a pharmaceutically acceptable carrier.

7. The pharmaceutical composition of claim 6, wherein the 5-HT1A agonist is vortioxetine, buspirone, flesinoxan, or an equivalent compound.

8. The pharmaceutical composition of claim 6, wherein the 5-HT1B modulator is zolmitriptan, elzasonan, or an equivalent compound.

9. The pharmaceutical composition of claim 6, wherein the mu-opioid receptor partial agonist or antagonist is buprenorphine, nalmefene, or an equivalent compound.

10. A method of identifying patients responsive to opioid tapering treatment comprising:
measuring baseline cortical receptor activity using neuroimaging techniques;
administering a test dose of a 5-HT1A agonist;
monitoring changes in cortical receptor-related activity;
correlating changes with clinical opioid withdrawal severity; and
adjusting opioid tapering protocols based on a patient neurobiological response profile.

11. The method of claim 10, wherein neuroimaging techniques include resting-state functional MRI, positron emission tomography, or electroencephalography.

12. A method of mitigating opioid withdrawal symptoms comprising co-administering a serotonergic agent and a mu-opioid receptor modulator during opioid tapering to reduce withdrawal severity and enhance treatment adherence.

13. A method of preventing opioid-induced hyperalgesia during tapering comprising administering a serotonergic agent to counteract opioid withdrawal-induced hyperexcitability in cortical pain-processing circuits.

14. A system for guiding opioid tapering comprising:
a database containing neuroimaging profiles of patients undergoing opioid tapering;
a processing unit configured to analyze receptor-related activity and predict optimal tapering protocols; and
a user interface for clinicians to input patient-specific data and receive personalized tapering recommendations.

15. The system of claim 14, wherein the processing unit employs machine learning algorithms to refine opioid tapering strategies based on patient response patterns over time.

16. A pharmaceutical composition for facilitating opioid tapering in patients undergoing long-term opioid therapy comprising:
a therapeutically effective amount of a serotonin receptor modulator;
a therapeutically effective amount of an opioid receptor modulator; and
a pharmaceutically acceptable carrier.

17. The pharmaceutical composition of claim 16, wherein the serotonin receptor modulator is a 5-HT1A agonist selected from the group consisting of vortioxetine, buspirone, flesinoxan, and tandospirone.

18. The pharmaceutical composition of claim 16, wherein the opioid receptor modulator is a partial agonist or antagonist selected from the group consisting of buprenorphine, nalmefene, and naltrexone.

19. The pharmaceutical composition of claim 16, wherein the composition is formulated for controlled-release administration to sustain therapeutic effects over an extended period.

20. The pharmaceutical composition of claim 16, wherein an administration regimen is adjusted based on patient-specific factors including opioid dependence severity, withdrawal symptoms, and neurobiological response to treatment.
